SEQUENCE LISTING (66 sequences)

SEQ ID NO: 1 - protein, 504 aa, Jatropha curcas
MLFFITVLFIFIALRIWKKSKANSTPNLPPGPNKLPLIGNVHNLVGDLPYHRLRDLSKKYGPIMHLQLGENTTVVISSPELAQEVMKTHDVNFAQRPFVLAGDIVSYKCKDIAFAPYGEYWRQLRKMCSLELLTAKRVQSFKSIREEEVSKLVESISSSSGSPINFSKMASSLTYAIISRAVCGKVSRGEEVFVPAVEKLVEAGRSISLADLYPSVKLFNALSVVRRRVEKIHGEVDKIIENIVIEHRERKRMAHAGINSKEEEDLVDVLLKFQENGDLDSYLSNDGIKAVILDMFIAGSDTSSTTIEWAISEMVKNPSIMEKAQAEVREVFGSKGKVDEADLHELNYLKLVIKETLRLHPAVPLLLPRQSREDCVIEGYNIATKSTVIVNAWAIARDPKYWDEAERFYPERFINSSIDFKGTNFEFIPFGAGRRMCPGMLFGLASVELPLAQLLYHFDWKLPGGQKPEDLDMSDDLDGTATRRHALYLTATPYLPSAVGKISR

SEQ ID NO: 2 - protein, 487 aa, Jatropha curcas
KKSKANSTPNLPPGPNKLPLIGNVHNLVGDLPYHRLRDLSKKYGPIMHLQLGENTTVVISSPELAQEVMKTHDVNFAQRPFVLAGDIVSYKCKDIAFAPYGEYWRQLRKMCSLELLTAKRVQSFKSIREEEVSKLVESISSSSGSPINFSKMASSLTYAIISRAVCGKVSRGEEVFVPAVEKLVEAGRSISLADLYPSVKLFNALSVVRRRVEKIHGEVDKIIENIVIEHRERKRMAHAGINSKEEEDLVDVLLKFQENGDLDSYLSNDGIKAVILDMFIAGSDTSSTTIEWAISEMVKNPSIMEKAQAEVREVFGSKGKVDEADLHELNYLKLVIKETLRLHPAVPLLLPRQSREDCVIEGYNIATKSTVIVNAWAIARDPKYWDEAERFYPERFINSSIDFKGTNFEFIPFGAGRRMCPGMLFGLASVELPLAQLLYHFDWKLPGGQKPEDLDMSDDLDGTATRRHALYLTATPYLPSAVGKISR

SEQ ID NO: 3 - DNA, 1515 bp, Jatropha curcas
atgcttttcttcatcaccgtactcttcattttcatcgcattaaggatatggaagaaatcaaaagccaactcaaccccaaatctaccaccaggaccaaacaaactacctctaatagggaacgttcacaatttagtcggcgatttaccctatcaccgcctaagagatctatccaagaaatacggacccattatgcaccttcagctcggcgaaaataccaccgtagtaatttcttcaccggaacttgctcaagaagttatgaaaacccatgacgtcaattttgctcaaaggccttttgtcctcgccggtgatatcgtaagctataaatgtaaagatatcgcatttgcgccttatggagaatattggcgacaattgcgaaagatgtgctccctcgagttattaactgcgaagcgtgtacagtcattcaaatcaatcagagaagaagaagtgtctaaactcgttgaatcgatatcttcaagctcaggatcgcctatcaattttagcaaaatggctagttcgttgacatatgctattatttcaagagctgtctgtggtaaagtatcgcgaggagaagaagtatttgtgccggctgttgaaaagttggttgaagcagggagaagtattagtcttgctgatttgtatccctctgttaaattgtttaatgctcttagtgttgtaaggcgtagagtagagaagatccatggggaagtagataagataattgaaaatattgtgatcgaacacagagagagaaaaagaatggcacatgctgggataaacagtaaggaggaagaagatcttgtagatgttcttttgaaatttcaagaaaatggggaccttgattcatatctatccaacgatggcatcaaagcagtaatcttggacatgttcatcgctggtagtgacacgtcatcaacaaccatagaatgggcaatatcagaaatggtgaaaaacccctcaataatggaaaaggcacaagcagaagtgagggaagtttttggttctaaaggaaaggtcgatgaagcagacttgcatgaactaaactacttgaaattggtgatcaaagaaactctgagattacacccagctgtcccattgttactcccaagacagagcagagaggattgtgtaattgaaggttataatatagctacgaaatctactgtcattgtgaatgcatgggctattgcgagggatccaaaatattgggatgaagctgagagattttatccagaaagattcattaatagttcaattgattttaaagggactaattttgaatttatcccatttggagctggaaggaggatgtgtcctggaatgttatttggtcttgcttccgttgagcttccacttgcacagttactatatcattttgattggaagcttcctggtggacagaagccagaagatcttgacatgtctgacgatcttgatggtacagcaacgagaagacatgctctatatttaactgccactccatatcttccttcagcagttggtaaaatctcaagatag

SEQ ID NO: 4 - DNA, 1889 bp, Jatropha curcas
atgtcgctgcaaccagcaattttacagggaaatacctgtaaacagtattttcatccattatcaagcatatcctctaccagatgggttggcaattgcaaccgtttcgcttttctttctccggctaagccaactgcaaacagagcaccgcaagcgtctttatcatcaaaactgcagccagtagttcgtctgctgactaaattccctgcttctggtttcttggccatgaatcaatctgttgatcaatttgcttcaactaccacaagtcttaccaaaatattcaacaaaataggaaaacctatccaatcatctccatttcttgtaagcgttcttcttttgatgtttatggcatcaaaaatacagaaccaacaagaagaagatgataactccataaatcttcctccaggaccatggagattacctttcataggtaacattcaccaacttgctggccccggtctaccccatcaccgtctaacagacttagccaaaacttacggacctgtaatgggtgttcaccttggcgaagtttacgctgttgttgtttcctccgcagaaacatccaaagaagtattaagaacgcaggatacaaatttcgctgaaagacctttagttaatgcagcgaaaatggtcctatataacagaaacgacattgtttttgggtcgtttggagatcaatggcgacaaatgagaaaaatctgcacattagaattacttagtgtaaaacgtgtgcagtcattcaaatcagtaagagaagaagagatgtcaagttttattaaatttctttcttcgaaatctggttcgccggtaaatcttacccatcatctgtttgttttgacaaactatattattgcaagaacttccattggtaagaaatgtaagaatcaagaagcgcttcttagaattatagacgacgtcgttgaggcgggagctggatttagtgttactgatgtctttccatcgtttgaagcgcttcatgtgattagtggagataagcataaatttgataaattgcatagagaaactgataagatacttgaagatatcataagtgaacataaagccgacagggcagtatcttccaagaaaagtgatggtgaagttgagaatcttcttgatgttcttttggatcttcaagaaaatggaaaccttcaatttcccttaacaaatgatgccatcaaaggagccattctggatacatttggcgcaggcagcgacacatcctcaaaaacagcagaatggacattatcggagctgatcaggaacccagaagcaatgagaaaagcacaagcagaaataaggagagttttcgatgaaacaggatatgttgatgaagacaaatttgaggaattaaaatacctgaaactagttgtgaaggaaactttgagattacatcctgctgtgccattaattccaagagaatgcagaggaaaaactaagattaatgggtatgacattttccccaagaccaaggtattggtgaacgtctgggcaatttcaagagatcctgcaatttggccagagcctgaaaagttcaatccagaaagattcatcgataatccgattgattataagatattaactgcgagctaacaccttttggtgcgggaaagagaatttgccctggaatgacattagggataacaaatcttgaacttttcctggcaaatttgctatatcattttgattggaaacttcctgacgggaagatgccagaggatcttgatatgagtgaatcatttggtggagcaattaaaagaaaaacagatctgaagttgattcctgttctggcgcgccctttgactccaagaaacgccaacagtggcaacactttcactacaacagacgccgactctcctgcatcaatgtgcccacacttaaaagcattatga

SEQ ID NO: 5 - DNA, 1512 bp, Jatropha curcas
atggaacaccaaatcctctcatttccagttcttttcagtttgcttctttttattctcgtcttactaaaagtatccaagaaattatacaaacatgactctaaacctccgcctggaccatggaaattacctttcataggtaaccttatccagctcgtcggtgacacacctcatcgccggttaacagccttggccaaaacttacggacctgtaatgggtgttcaacttgggcaagttcctttccttgtcgtgtcctcgccggaaacagctaaagaagtaatgaaaatacaagatcccgtttttgcagaacgaccgcttgtccttgcaggagaaatagtgctttataaccgaaatgacatcgtttttgggtcgtacggagatcagtggaggcaaatgagaaaattttgcacgttggaattacttagcacaaaacgagtacagtcgttccgacccgtgagagaagaagaagttgcatcttttgtaaaacttatgcgtacaaagaaaggaactcctgttaatcttactcatgctttatttgctttaacaaattctatagttgcaagaaatgctgttggtcataaaagcaaaaaccaagaggcgttgttagaagttattgatgacatagttgtatcaggaggaggtgttagtatagttgatatctttccttccctacaatggcttcctactgccaagagggaaagatcaagaatttggaaattgcaccaaaatacagatgagattctcgaagatatcttacaagagcatagagctaaaagacaggcgacagcttccaagaattgggataggagcgaagctgataatcttcttgatgttcttttggatcttcaacagagcggaaatcttgatgttcctttaactgatgtcgccatcaaagcagcaattattgatatgtttggtgctggaagcgacacatcctcaaaaactgcagaatgggcaatggctgagttgatgaggaatccagaagtaatgaagaaagcacaagaagaattgcggaatttctttggtgaaaatggaaaggttgaggaagcaaaacttcacgaattaaaatggataaagttaattattaaagaaacattgagattacatcctgcagtggctgtaattccaagggtttgtagggaaaagactaaagtttatggatatgacgttgagcctggcactcgggttttcattaacgtgtggtcaatcggaagagatcctaaagtttggagtgaagctgagagattcaagccggagagatttattgatagcgcaattgattacaggggtcttaattttgaactgattccatttggagcaggaaaaagaatatgccctggaatgaccttaggaatggctaatctggagattttccttgcaaacttgctatatcattttgactggaaatttcctaaaggagtaactgcagaaaatcttgacatgaatgaagcttttggaggagctgtcaaaagaaaagtagaccttgaattgatccccattccattccgtccctaa

SEQ ID NO: 6 - DNA, 1806 bp, Jatropha curcas
atggcaatgcaacctgcaattgttcaagcaaactcccaaaaacaaatccttactactccgttcttattaagcacacctagtactaagcttaacgacagtcgttttgcttccttttccttggctaagccaacaacttttagaaaacttaaagcatgtgcatcaacaaaatctgagacagaagctcgtcccttagcctactttcctcctactgtatggggcgatcgacttgcttctcttaccttcaatcaaccggcatttgaattattaagtaaacaagtagagttgttgaacgaaaagattaaaaaagaaatgttaaacgtctctacaagtgatttagcagagaaaatcattttgatcgactcattgtgccgtctcggagtatcatatcactttgaggaggagattcaagagaatctaactaggattttcaatacgcaacctaatttccttaatgaaaaagattatgatctcttcactgttgctgtaatatttcgagtatttagacagcatggtttcaaaatctcttctgatgtgttcaacaagttcaaggatagtgatggtaagttcaaggaatccctactaaatgatatcaaaggcatactgagcctttttgaagctacacatgtgagcatgcctaatgaacccattttagatgaggccttagctttcaccaaggctttcttggaatcctctgcggttaagtcattccctaatttcgcaaagcatataagcagtgcactagagcagccggtacacaaaggcataccaaggctagaggcaagaaaatatattgatttatacgaggtcgatgaaagtcgaaatgaaactgtactagagcttgcaaagttggattttaacagagtgcagttgctacaccaagaagaattaagtcaattttcaaagtggtggaagagtttgaatattagtgcagaggtcccatatgcaagaaacagaatggcagagattttcttttgggcggtttctatgtattttaaacctcaatatgcaaaggctagaatgatcgtctccaaagtcgtattacttatctcactcatagacgatacaattgatgcatatgccactattgatgaaatccaccgtgttgcagatgcaatcgaaaggtgggatatgagattagtcgaccaactgccaaattacatgaaagtaatctatagattaattatcaacacatttgatgaatttgagaaagatttggaagcagaaggaaagtcctacagcgtcaagtatggaagggaagcgtatcaagagctagtgagaggctattacttggaggcgatatggaaggcggacggaaaagtgccgtcgtttgatgagtacatatataacggaggtgtgaccactggattgcctcttgttgccactgtatcatttatgggagtaaaagaaattaaaggaactaaagcattccaatggctaaaaacctaccccaaactcaatcaagctggaggtgaatttatccgtctggtgaatgatgtaatgtctcatgagactgagcaagatagaggacatgtagcgtcttgcatcgattgctacatgaagcaatatggtgtttcaaaagaggaggcagttgaagagatccagaaaatggctacaaatgaatggaagaaattaaatgagcaactcatcgtgcggtccacagaagttgtgccagtgaatcttttaatgcgaatcgttaatcttgtccgcctaacagatgtgagttacaagtatggagatggatacacggactcttcacaactgaaagaatatgtgaaaggattgttcattgaacctattgccacttga

SEQ ID NO: 7 - DNA, 20 bp, artificial sequence
  primer
cccagaaggaagtatgcccg

SEQ ID NO: 8 - DNA, 20 bp, artificial sequence
  primer
ctttgcaagttgcccaacga

SEQ ID NO: 9 - DNA, 21 bp, artificial sequence
  primer
ctccaagtcccagaaggaagt

SEQ ID NO: 10 - DNA, 22 bp, artificial sequence
  primer
cgggaaaatctaggctgagtgt

SEQ ID NO: 11 - DNA, 22 bp, artificial sequence
  primer
gcagtgttgctgaatatgaggc

SEQ ID NO: 12 - DNA, 22 bp, artificial sequence
  primer
tcccgcaatgaatcttgtctga

SEQ ID NO: 13 - DNA, 20 bp, artificial sequence
  primer
agctcgcaggctaccaattt

SEQ ID NO: 14 - DNA, 20 bp, artificial sequence
  primer
cttctttggccatttccggc

SEQ ID NO: 15 - DNA, 20 bp, artificial sequence
  primer
ctgggcatccttttgcacca

SEQ ID NO: 16 - DNA, 18 bp, artificial sequence
  primer
tcttgaagtctggcggcg

SEQ ID NO: 17 - DNA, 20 bp, artificial sequence
  primer
taacaggaaggcggcagttc

SEQ ID NO: 18 - DNA, 20 bp, artificial sequence
  primer
ctgccagccccaaacatttc

SEQ ID NO: 19 - DNA, 23 bp, artificial sequence
  primer
tgctgggataaacagtaaggagg

SEQ ID NO: 20 - DNA, 20 bp, artificial sequence
  primer
atgacgtgtcactaccagcg

SEQ ID NO: 21 - DNA, 20 bp, artificial sequence
  primer
cagctcggcgaaattaccac

SEQ ID NO: 22 - DNA, 20 bp, artificial sequence
  primer
gtgcgagtgcgatatctgtg

SEQ ID NO: 23 - DNA, 20 bp, artificial sequence
  primer
gggtttgagcgaacagcaag

SEQ ID NO: 24 - DNA, 20 bp, artificial sequence
  primer
agcaaggtacaaagcagcct

SEQ ID NO: 25 - DNA, 20 bp, artificial sequence
  primer
ctcaaacccagcttttgccc

SEQ ID NO: 26 - DNA, 20 bp, artificial sequence
  primer
tcgttggggttattggcaca

SEQ ID NO: 27 - DNA, 20 bp, artificial sequence
  primer
atggcgggttcggatcttac

SEQ ID NO: 28 - DNA, 20 bp, artificial sequence
  primer
gacattgcttgttgagccgt

SEQ ID NO: 29 - DNA, 20 bp, artificial sequence
  primer
gctactgcgtacctgctgat

SEQ ID NO: 30 - DNA, 20 bp, artificial sequence
  primer
agggccactaaaaactcggg

SEQ ID NO: 31 - DNA, 20 bp, artificial sequence
  primer
aacataaagccgacagggca

SEQ ID NO: 32 - DNA, 20 bp, artificial sequence
  primer
ctgcctgcgccaaatgtatc

SEQ ID NO: 33 - DNA, 20 bp, artificial sequence
  primer
cctagtggcaagctgaacga

SEQ ID NO: 34 - DNA, 21 bp, artificial sequence
  primer
tggacgagtgtctgtctctga

SEQ ID NO: 35 - DNA, 20 bp, artificial sequence
  primer
acatgtttaatggcggggtt

SEQ ID NO: 36 - DNA, 20 bp, artificial sequence
  primer
ttcgcctccagcttgattga

SEQ ID NO: 37 - DNA, 20 bp, artificial sequence
  primer
ggtccacagaagttgtgcca

SEQ ID NO: 38 - DNA, 21 bp, artificial sequence
  primer
tcagttgtgaagagtccgtgt

SEQ ID NO: 39 - DNA, 20 bp, artificial sequence
  primer
ttgggataggagcgaagctg

SEQ ID NO: 40 - DNA, 20 bp, artificial sequence
  primer
tcgcttccagcaccaaacat

SEQ ID NO: 41 - DNA, 20 bp, artificial sequence
  primer
ctgatcgaccgcttgtcctt

SEQ ID NO: 42 - DNA, 20 bp, artificial sequence
  primer
ctccgtacagcccaaaacct

SEQ ID NO: 43 - DNA, 22 bp, artificial sequence
  primer
tgccatccaggccgttctatct

SEQ ID NO: 44 - DNA, 23 bp, artificial sequence
  primer
ggaggatagcatgtggaagagcg

SEQ ID NO: 45 - DNA, 43 bp, artificial sequence
  primer
ctgcccaaattcgcgaccggtaaaaatggcaatgcaacctgca

SEQ ID NO: 46 - DNA, 30 bp, artificial sequence
  primer
ttgctcacccatacagtaggaggaaagtag

SEQ ID NO: 47 - DNA, 26 bp, artificial sequence
  primer
ctgtatgggtgagcaagggcgaggag

SEQ ID NO: 48 - DNA, 41 bp, artificial sequence
  primer
gaaaccagagttaaaggccttacttgtacagctcgtccatg

SEQ ID NO: 49 - DNA, 43 bp, artificial sequence
  primer
ctgcccaaattcgcgaccggtaaaaatgtcgctgcaaccagca

SEQ ID NO: 50 - DNA, 37 bp, artificial sequence
  primer
ttgctcacgaatattttggtaagacttgtggtagttg

SEQ ID NO: 51 - DNA, 28 bp, artificial sequence
  primer
caaaatattcgtgagcaagggcgaggag

SEQ ID NO: 52 - DNA, 41 bp, artificial sequence; identical to SEQ ID NO: 48
  primer

SEQ ID NO: 53 - DNA, 43 bp, artificial sequence
  primer
ctgcccaaattcgcgaccggtaaaaatggaacaccaaatcctc

SEQ ID NO: 54 - DNA, 26 bp, artificial sequence
  primer
ttgctcacgaaaggaacttgcccaag

SEQ ID NO: 55 - DNA, 27 bp, artificial sequence
  primer
gttcctttcgtgagcaagggcgaggag

SEQ ID NO: 56 - DNA, 41 bp, artificial sequence; identical to SEQ ID NO: 48
  primer

SEQ ID NO: 57 - DNA, 43 bp, artificial sequence
  primer
aaaaggtctcaccggaaaaatgcttttcttcatcaccgtactc

SEQ ID NO: 58 - DNA, 41 bp, artificial sequence
  primer
aaaaggtctcatcgactatcttgagattttaccaactgctg

SEQ ID NO: 59 - DNA, 39 bp, artificial sequence
  primer
aaaaaccggtaaaaatgtcgctgcaaccagcaattttac

SEQ ID NO: 60 - DNA, 35 bp, artificial sequence
  primer
aaaactcgagtcataatgcttttaagtgtgggcac

SEQ ID NO: 61 - DNA, 48 bp, artificial sequence
  primer
tattctgcccaaattcgcgaaaaaatggaacaccaaatcctctcattt

SEQ ID NO: 62 - DNA, 42 bp, artificial sequence
  primer
tgaaaccagagttaaaggccttagggacggaatggaatgggg

SEQ ID NO: 63 - DNA, 48 bp, artificial sequence
  primer
tattctgcccaaattcgcgaccggtaaaaatggcttcttctgcatttg

SEQ ID NO: 64 - DNA, 45 bp, artificial sequence
  primer
tgaaaccagagttaaaggcctcgagtcaaaacagagcttcccttg

SEQ ID NO: 65 - DNA, 48 bp, artificial sequence
  primer
tattctgcccaaattcgcgaccggtaaaaatggcttcagtgactctag

SEQ ID NO: 66 - DNA, 47 bp, artificial sequence
  primer
tgaaaccagagttaaaggcctcgagtcagttctgtctataggcaatg

(19) United States
(12) Patent Application Publication
(10) Pub. No.: US 2019/0106684 A1
(43) Pub. Date: Apr. 11, 2019

(54) MODIFIED CELL

(71) Applicant: The University of York, York (GB)

(72) Inventors: Ian Graham, York (GB); Andrew King, Whitstable (GB)

(73) Assignee: The University of York, York (GB)

(21) Appl. No.: 16/082,383

(22) PCT Filed: Mar. 13, 2017
(86) PCT No.: PCT/GB2017/050679

§ 371 Date: Sep. 5, 2018

(30) Foreign Application Priority Data
Mar. 16, 2016 (GB) 1604427.3

(51) Int. Cl. C12N 9/04; C12N 9/88; C12N 15/82; C12P 17/02; C12P 7/26

(52) U.S. Cl. CPC C12N 9/0006; C12N 9/88; C12P 7/26; C12N 15/8243; C12P 17/02; C12Y 402/03008

(57) ABSTRACT

The present disclosure relates to nucleic acids that encode enzyme activities involved in the synthesis of lathyranes, intermediates in the synthesis of lathyranes and also compounds derived from lathyranes such as tiglianes, daphnanes and ingenanes; cells transformed with the nucleic acid molecules and vectors comprising the nucleic acid molecules.

### CROSS REFERENCE TO RELATED APPLICATIONS

This is the U.S. National Stage of International Application No. PCT/GB2017/050679, filed Mar. 13, 2017, which was published in English under PCT Article 21(2), which in turn claims the benefit of Great Britain Application No. 1604427.3, filed Mar. 16, 2016.

### FIELD OF THE INVENTION

The present disclosure relates to nucleic acids that encode enzyme activities involved in the synthesis of lathyranes, intermediates in the synthesis of lathyranes and also compounds derived from lathyranes such as tiglianes, daphnanes and ingenanes; cells transformed with the nucleic acid molecules and vectors comprising the nucleic acid molecules.

### BACKGROUND TO THE INVENTION

Terpenes or terpenoids are a structurally diverse and a very large group of organic compounds commonly found in plants ranging from essential and universal primary metabolites such as sterols, carotenoids and hormones to more complex and unique secondary metabolites. Terpenes are hydrocarbons assembled of five carbon terpene or isoprene subunits providing the carbon skeleton. Terpenoids are modified terpenes which typically comprise also oxygen. Terpenoids are classified accordingly to the length of the isoprene units as for example hemiterpenoids consisting of one, monoterpenoids consisting of two, sesquiterpenoids consisting of three and diterpenoids consisting of four isoprene units.

Diterpenes form the basis for many biologically important compounds such as retinol, retinal, and phytol and some compounds have shown anti-microbial and anti-inflammatory properties. A large number of diterpenes have been isolated from plants belonging to the family of Euphorbiaceae. The Euphorbiaceae or spurge family is a large family of flowering plants found all over the world, with some synthesising compounds of considerable biological activity such as ingenol mebutate (Euphorbia peplus), resiniferatoxin (E. resinifera), prostratin (E. cornigera), jatrophanes and lathyranes (Jatropha sp. and Euphorbia sp.), jatropholones, (Jatropha sp.), rhamnofolanes (Jatropha sp.) and jatrophone (Jatropha sp.).

The Euphorbiaceae produce a diverse range of casbene derived diterpenoids^{1,2}, many of which are providing interesting leads in the development of new pharmaceuticals. These include the lathyranes which are inhibitors of ABC transporters responsible for the efflux of chemotherapy drugs in multidrug-resistant (MDR) cancers^{3,4} as wells as fungal⁵ and protozoal⁶ pathogens. The lathyranes are also precursors of many other active diterpenoids including ingenol mebutate, a licenced pharmaceutical used for the treatment of actinic keratosis⁷, prostratin, a lead compound for the treatment of latent HIV infections⁸, and resiniferatoxin, an ultrapotent capsaicin analog which is currently in clinical trial for the treatment of cancer-related intractable pain⁹. Although the relationship between casbene and lathyrane structure was noted several decades ago¹⁰, the mechanism leading to the ring closure required to convert the 14:3 casbane ring into the 5:11:3 lathyrane ring system has not previously been reported

In co-pending PCT application WO2015/104553 is disclosed genes encoding enzymes involved in diterpenoid biosynthesis, including casbene-5-oxidases. This disclosure relates to the identification and characterisation of additional enzyme activities involved in the biosynthesis of diterpenes, such as lathyranes, from geranylgeranyl pyrophosphate via the 9-oxidation of the casbene skeleton, as occurs for example in the biosynthesis of jolkinol C and epi-jolkinol C. The conversion of casbene to a lathyrane skeleton involves a co-ordinated cytochrome P450 mediated intramolecular carbon-carbon ring closure.

### STATEMENTS OF INVENTION

According to an aspect of the invention there is provided an isolated cell transformed or transfected with an expression vector adapted to express a nucleic acid molecule comprising a nucleotide sequence as set forth in SEQ ID NO: 3, or a nucleotide sequence that has at least 50% sequence identity to the nucleotide sequence set forth in SEQ ID NO: 3 and encodes a polypeptide that has casbene 9-oxidase activity.

Substrates for the casbene 9-oxidase according to the invention are varied, for example, casbene, 9-hydroxy casbene, 5-hydroxy-casbene, 5-keto-casbene and 6-hydroxy-5-keto-casbene.

In an embodiment of the invention said isolated cell is further transformed with an expression vector adapted to express a nucleic acid molecule comprising nucleotide sequence as set forth in SEQ ID NO: 6, or a nucleotide sequence that has at least 50% sequence identity to the nucleotide sequence set forth in SEQ ID NO: 6 and encodes a polypeptide that has casbene synthase activity.

In an alternative embodiment of the invention said isolated cell is further transformed with an expression vector adapted to express a nucleic acid molecule comprising nucleotide sequence as set forth in SEQ ID NO: 4, or a nucleotide sequence that has at least 50% sequence identity to the nucleotide sequence set forth in SEQ ID NO: 4 and encodes a polypeptide that has casbene-5, 6-oxidase activity

In a further alternative embodiment of the invention said isolated cell is further transformed with an expression vector adapted to express a nucleic acid molecule comprising nucleotide sequence as set forth in SEQ ID NO: 5 or a nucleotide sequence that has at least 50% sequence identity to the nucleotide sequence set forth in SEQ ID NO: 5 and encodes a polypeptide that has casbene-5, 6-oxidase activity

In an embodiment of the invention there is provided an isolated cell transformed with at least one vector comprising a nucleotide sequence from the group consisting of:
i) a nucleic acid molecule comprising nucleotide sequence as set forth in SEQ ID NO: 3, or a nucleotide sequence that has at least 50% sequence identity to the nucleotide sequence set forth in SEQ ID NO: 3 and encodes a polypeptide that has casbene oxidase activity; and
ii) a nucleic acid molecule comprising nucleotide sequence as set forth in SEQ ID NO: 6, or a nucleotide sequence that has at least 50% sequence identity to the nucleotide sequence set forth in SEQ ID NO: 6 and encodes a polypeptide that has casbene synthase activity.

In an alternative embodiment of the invention there is provided an isolated cell transformed with at least one vector comprising a nucleotide sequence from the group consisting of:
i) a nucleic acid molecule comprising nucleotide sequence as set forth in SEQ ID NO: 3, or a nucleotide sequence that has at least 50% sequence identity to the nucleotide sequence set forth in SEQ ID NO: 3 and encodes a polypeptide that has casbene oxidase activity; and
ii) a nucleic acid molecule comprising nucleotide sequence as set forth in SEQ ID NO: 6, or a nucleotide sequence that has at least 50% sequence identity to the nucleotide sequence set forth in SEQ ID NO: 6 and encodes a polypeptide that has casbene synthase activity; and
iii) a nucleic acid molecule comprising nucleotide sequence as set forth in SEQ ID NO: 4, or a nucleotide sequence that has at least 50% sequence identity to the nucleotide sequence set forth in SEQ ID NO: 4 and encodes a polypeptide that has casbene 5,6-oxidase activity.

In a further alternative embodiment of the invention there is provided an isolated cell transformed with at least one vector comprising a nucleotide sequence from the group consisting of:
i) a nucleic acid molecule comprising nucleotide sequence as set forth in SEQ ID NO: 3, or a nucleotide sequence that has at least 50% sequence identity to the nucleotide sequence set forth in SEQ ID NO: 3 and encodes a polypeptide that has casbene oxidase activity; and
ii) a nucleic acid molecule comprising nucleotide sequence as set forth in SEQ ID NO: 6, or a nucleotide sequence that has at least 50% sequence identity to the nucleotide sequence set forth in SEQ ID NO: 6 and encodes a polypeptide that has casbene synthase activity; and
iii) a nucleic acid molecule comprising nucleotide sequence as set forth in SEQ ID NO: 5, or a nucleotide sequence that has at least 50% sequence identity to the nucleotide sequence set forth in SEQ ID NO: 5 and encodes a polypeptide that has casbene 5,6-oxidase activity.

In an embodiment of the invention said isolated cell transformed or transfected with an expression vector adapted to express a nucleic acid molecule encoding a polypeptide comprising or consisting of an amino acid sequence as set forth in SEQ ID NO: 1 or 2, or an amino acid sequence that is at least 50% identical to the amino acid sequence set forth in SEQ ID NO: 1 or 2 and which has casbene-9-oxidase activity.

In an embodiment of the invention said nucleic acid molecule comprises or consists of a nucleotide sequence that has at least 55%, 60%, 65%, 70%, 75%, 80%, 85%, 90%, 95%, 96%, 97%, 98% or 99% nucleotide sequence identity to the nucleotide sequences set forth in SEQ ID NO: 3, 4, 5 or 6 over the full length sequence or over the full length sequence of the amino acid sequence set forth in SEQ ID NO: 1 or 2.

In an embodiment of the invention said isolated cell is transformed or transfected with an expression vector adapted to express a nucleic acid molecule encoding a polypeptide comprising or consisting of an amino acid sequence as set forth in SEQ ID NO: 1 or 2.

In an embodiment of the invention said isolated cell is a plant cell.

In an alternative embodiment of the invention said cell is a microbial cell.

In an embodiment of the invention said microbial cell is a bacterial cell.

In an embodiment of the invention said microbial cell is a fungal cell, for example a yeast cell.

In a further alternative embodiment of the invention said cell is an algal cell.

If microbial cells, for example bacterial or yeast cells are used as organisms in the process according to the invention they are grown or cultured in the manner with which the skilled worker is familiar, depending on the host organism. As a rule, microorganisms are grown in a liquid medium comprising a carbon source, usually in the form of sugars, a nitrogen source, usually in the form of organic nitrogen sources such as yeast extract or salts such as ammonium sulfate, trace elements such as salts of iron, manganese and magnesium and, if appropriate, vitamins, at temperatures of between 0° C. and 100° C., preferably between 10° C. and 60° C., while gassing in oxygen.

The pH of the liquid medium can either be kept constant, that is to say regulated during the culturing period, or not. The cultures can be grown batchwise, semi-batchwise or continuously. Nutrients can be provided at the beginning of the fermentation or fed in semi-continuously or continuously. The diterpenoids produced can be isolated from the organisms as described above by processes known to the skilled worker, for example by extraction, distillation, crystallization, if appropriate precipitation with salt, and/or chromatography. To this end, the organisms can advantageously be disrupted beforehand. In this process, the pH value is advantageously kept between pH 4 and 12, preferably between pH 6 and 9, especially preferably between pH 7 and 8.

The culture medium to be used must suitably meet the requirements of the strains in question. Descriptions of culture media for various microorganisms can be found in the textbook “Manual of Methods for General Bacteriology” of the American Society for Bacteriology (Washington D.C., USA, 1981).

As described above, these media which can be employed in accordance with the invention usually comprise one or more carbon sources, nitrogen sources, inorganic salts, vitamins and/or trace elements.

Preferred carbon sources are sugars, such as mono-, di- or polysaccharides. Examples of carbon sources are glucose, fructose, mannose, galactose, ribose, sorbose, ribulose, lactose, maltose, sucrose, raffinose, starch or cellulose. Sugars can also be added to the media via complex compounds such as molasses or other by-products from sugar refining. The addition of mixtures of a variety of carbon sources may also be advantageous. Other possible carbon sources are oils and fats such as, for example, soya oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol, and/or organic acids such as, for example, acetic acid and/or lactic acid.

Nitrogen sources are usually organic or inorganic nitrogen compounds or materials comprising these compounds. Examples of nitrogen sources comprise ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as corn steep liquor, soya meal, soya protein, yeast extract, meat extract and others. The nitrogen sources can be used individually or as a mixture.

Inorganic salt compounds which may be present in the media comprise the chloride, phosphorus and sulfate salts of calcium, magnesium, sodium, cobalt, molybdenum, potassium, manganese, zinc, copper and iron.

Inorganic sulfur-containing compounds such as, for example, sulfates, sulfites, dithionites, tetrathionates, thiosulfates, sulfides, or else organic sulfur compounds such as mercaptans and thiols may be used as sources of sulfur for the production of sulfur-containing fine chemicals, in particular of methionine.

Phosphoric acid, potassium dihydrogen phosphate or dipotassium hydrogen phosphate or the corresponding sodium-containing salts may be used as sources of phosphorus. Chelating agents may be added to the medium in order to keep the metal ions in solution. Particularly suitable chelating agents comprise dihydroxyphenols such as catechol or protocatechuate and organic acids such as citric acid.

The fermentation media used according to the invention for culturing microorganisms usually also comprise other growth factors such as vitamins or growth promoters, which include, for example, biotin, riboflavin, thiamine, folic acid, nicotinic acid, pantothenate and pyridoxine. Growth factors and salts are frequently derived from complex media components such as yeast extract, molasses, corn steep liquor and the like. It is moreover possible to add suitable precursors to the culture medium. The exact composition of the media compounds heavily depends on the particular experiment and is decided upon individually for each specific case. Information on the optimization of media can be found in the textbook “Applied Microbiol. Physiology, A Practical Approach” (Editors P. M. Rhodes, P. F. Stanbury, IRL Press (1997) pp. 53-73, ISBN 0 19 963577 3). Growth media can also be obtained from commercial suppliers, for example Standard 1 (Merck) or BHI (brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture temperature is normally between 15° C. and 45° C., preferably at from 25° C. to 40° C., and may be kept constant or may be altered during the experiment. The pH of the medium should be in the range from 5 to 8.5, preferably around 7.0. The pH for cultivation can be controlled during cultivation by adding basic compounds such as sodium hydroxide, potassium hydroxide, ammonia and aqueous ammonia or acidic compounds such as phosphoric acid or sulfuric acid. Foaming can be controlled by employing antifoams such as, for example, fatty acid polyglycol esters. To maintain the stability of plasmids it is possible to add to the medium suitable substances having a selective effect, for example antibiotics. Aerobic conditions are maintained by introducing oxygen or oxygen-containing gas mixtures such as, for example, ambient air into the culture. The temperature of the culture is normally 20° C. to 45° C. and preferably 25° C. to 40° C. The culture is continued until formation of the desired product is at a maximum. This aim is normally achieved within 10 to 160 hours.

The fermentation broth can then be processed further. The biomass may, according to requirement, be removed completely or partially from the fermentation broth by separation methods such as, for example, centrifugation, filtration, decanting or a combination of these methods or be left completely in said broth. It is advantageous to process the biomass after its separation. However, the fermentation broth can also be thickened or concentrated without separating the cells, using known methods such as, for example, with the aid of a rotary evaporator, thin-film evaporator, falling-film evaporator, by reverse osmosis or by nanofiltration. Finally, this concentrated fermentation broth can be processed to obtain the diterpenoids present therein.

According to an aspect of the invention there is provided a plant transformed with a nucleic acid transcription cassette comprising a nucleotide sequence selected from the group consisting of:
i) a nucleotide sequence as set forth in SEQ ID NO: 3; or
ii) a nucleotide sequence that has at least 50% sequence identity to the nucleotide sequence set forth in SEQ ID NO: 3 and encodes a polypeptide that has casbene 9-oxidase activity.

In an embodiment of the invention said plant further comprises a transcription cassette comprising a nucleotide sequence selected from the group consisting of:
i) a nucleotide sequence as set forth in SEQ ID NO: 6; or
ii) a nucleotide sequence that has at least 50% sequence identity to the nucleotide sequence set forth in SEQ ID NO: 6 and encodes a polypeptide that has casbene synthase activity.

In an embodiment of the invention said plant further comprises a transcription cassette comprising a nucleotide sequence selected from the group consisting of:
i) a nucleotide sequence as set forth in SEQ ID NO: 4, or
ii) a nucleotide sequence that has at least 50% sequence identity to the nucleotide sequence set forth in SEQ ID NO: 4 and encodes a polypeptide that has casbene-5,6-oxidase activity.

In an embodiment of the invention said plant further comprises a transcription cassette comprising a nucleotide sequence selected from the group consisting of:
i) a nucleotide sequence as set forth in SEQ ID NO: 5; or
ii) a nucleotide sequence that has at least 50% sequence identity to the nucleotide sequence set forth in SEQ ID NO: 5 and encodes a polypeptide that has casbene-5,6-oxidase.

Plants according to the invention can be selected from: corn (Zea mays), canola (Brassica napus, Brassica rapa ssp.), alfalfa (Medicago sativa), rice (Oryza sativa), rye (Secale cerale), sorghum (Sorghum bicolor, Sorghum vulgare), sunflower (helianthus annuas), wheat (Tritium aestivum), soybean (Glycine max), tobacco (Nicotiana tabacum), potato (Solanum tuberosum), peanuts (Arachis hypogaea), cotton (Gossypium hirsutum), sweet potato (lopmoea batatus), cassava (Manihot esculenta), coffee (Cofea spp.), coconut (Cocos nucifera), pineapple (Anana comosus), citris tree (Citrus spp.) cocoa (Theobroma cacao), tea (Camellia senensis), banana (Musa spp.), avacado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), oats, barley, vegetables and ornamentals.

Preferably, plants of the present invention are crop plants (for example, cereals and pulses, maize, wheat, potatoes, tapioca, rice, sorghum, millet, cassava, barley, pea, and other root, tuber or seed crops. Important seed crops are oil-seed rape, sugar beet, maize, sunflower, soybean, and sorghum). Horticultural plants to which the present invention may be applied may include lettuce, endive, and vegetable brassicas including cabbage, broccoli, and cauliflower, and carnations and geraniums. The present invention may be applied in tobacco, cucurbits, carrot, strawberry, sunflower, tomato, pepper, chrysanthemum.

Grain plants that provide seeds of interest include oil-seed plants and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

In preferred embodiment of the invention said plant is from the Soanaceae family (e.g., Nicotiana tabacum or Nicotiana bethamiana).

In a preferred embodiment of the invention said transcription cassette is part of a vector adapted for expression in a plant cell.

According to an aspect of the invention there is provided a cell culture comprising a cell according to the invention.

According to a further aspect of the invention there is provided a process or the manufacture of a lathyrane diterpene, or intermediates thereof, comprising the steps:

i) providing a cell culture according to the invention wherein cells comprised in the culture express a casbene 9-oxidase [SEQ ID NO: 3], and cell culture medium supplemented with a compound selected from the group consisting of casbene, 6-hydroxy-5-keto-casbene, 5-keto-casbene, 5-hydroxycasbene or 9-hydroxy-casbene;
ii) culturing said cells in said culture; and optionally
iii) isolating or purifying synthesized compounds from the cells and/or cell culture medium.

According to an aspect of the invention there is provided a process for the manufacture of at least 9-keto casbene comprising the steps:
i) providing a cell culture according to the invention wherein cells comprised in the culture contain an endogenous pool of geranylgeranyl disphosphate and express a casbene-9-oxidase [SEQ ID NO: 3] and a casbene synthase [SEQ ID NO: 6];
ii) culturing said cells in said cell culture; and optionally
iii) isolating or purifying 9-keto casbene from the cells or cell culture medium.

According to a further aspect of the invention there is provided a process or the manufacture of a lathyrane diterpene, or intermediates thereof, comprising the steps:
i) providing a cell culture according to the invention wherein cells comprised in the culture contain an endogenous pool of geranylgeranyl disphosphate and express a casbene 9-oxidase [SEQ ID NO: 3], a casbene synthase [SEQ ID NO: 6] and a casbene 5,6-oxidase [SEQ ID NO: 4];
ii) culturing said cells in said culture; and optionally
iii) isolating or purifying synthesized compounds from the cells and/or cell culture medium.

According to a further aspect of the invention there is provided a process or the manufacture of a lathyrane diterpene, or intermediates thereof, comprising the steps:
i) providing a cell culture according to the invention wherein cells comprised in the culture contain an endogenous pool of geranylgeranyl disphosphate and express a casbene 9-oxidase [SEQ ID NO: 3], a casbene synthase [SEQ ID NO: 6] and a casbene 5,6-oxidase [SEQ ID NO: 5];
ii) culturing said cells in said culture; and optionally
iii) isolating or purifying synthesized compounds from the cells and/or cell culture medium.

In a preferred method of the invention said compound is jolkinol C or epi-jolkinol C.

According to an aspect of the invention there is provided an isolated polypeptide comprising or consisting of an amino acid sequence as set forth in SEQ ID NO: 2, or an amino acid sequence that has greater than 96% amino acid sequence identity to SEQ ID NO: 2.

In an embodiment of the invention said polypeptide has at least 97%, 98% or 99% amino acid sequence identity to SEQ ID NO: 2.

According to an aspect of the invention there is provided a nucleic acid molecule encoding a polypeptide according to the invention.

In an embodiment of the invention said nucleic acid molecule is part of an expression vector adapted for expression of said nucleic acid molecule.

In an embodiment of the invention said vector is adapted for expression in a microbial host cell.

According to an aspect of the invention there is provided the use of a polypeptide encoded by a nucleic acid molecule comprising a nucleotide sequence set forth in SEQ ID NO 3, or a nucleic acid molecule comprising a nucleotide sequence that has at least 50% sequence identity to the nucleotide sequence set forth in SEQ ID NO: 3 and encodes a polypeptide that has casbene 9-oxidase activity in the transformation of casbene diterpenes, such as 6-hydroxy-5-keto-casbene to lathyrane, lathyrane diterpene intermediates and lathyrane diterpenes.

In a preferred embodiment of the invention said nucleic acid molecule is expressed by an isolated cell.

In a preferred embodiment of the invention said isolated cell is selected from the group a plant cell, a microbial cell, a fungal cell or an algal cell.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

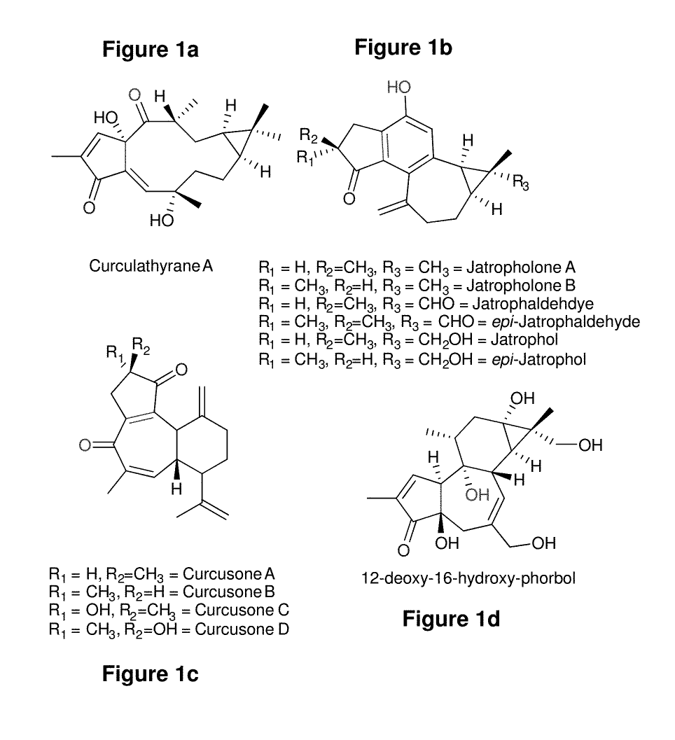

FIGS. 1a-1d: Diterpenoids of the (a) lathyrane (b) jatropholane (c) rhamnofolane and (d) tigliane class which have been isolated from J. curcas. The red oxygen atom highlighted on each of the molecules corresponds to the 5-position of casbene, whereas the blue oxygen atom corresponds to the 9-position of casbene. The carbon-carbon bond highlighted in green corresponds to the 6, 10-positions of casbene.

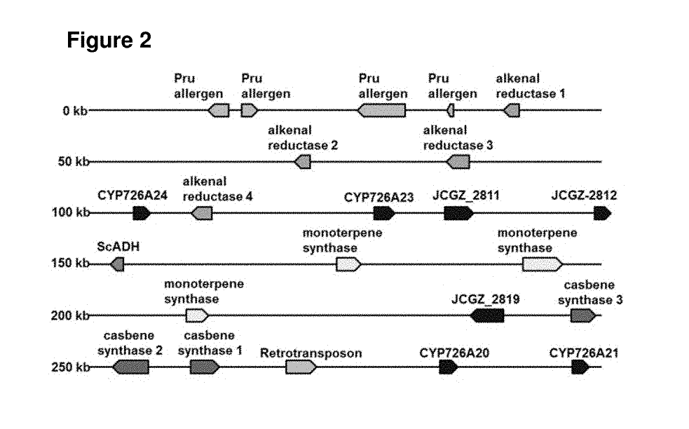

FIG. 2: A diterpenoid biosynthesis gene cluster. The diagram corresponds to a 300 kbp region present on scaffold 123 of the J. curcas genome (Genbank accession NW_012124159). Different classes of enzymes have been colour-coded, e.g., cytochrome P450 genes are shown in blue.

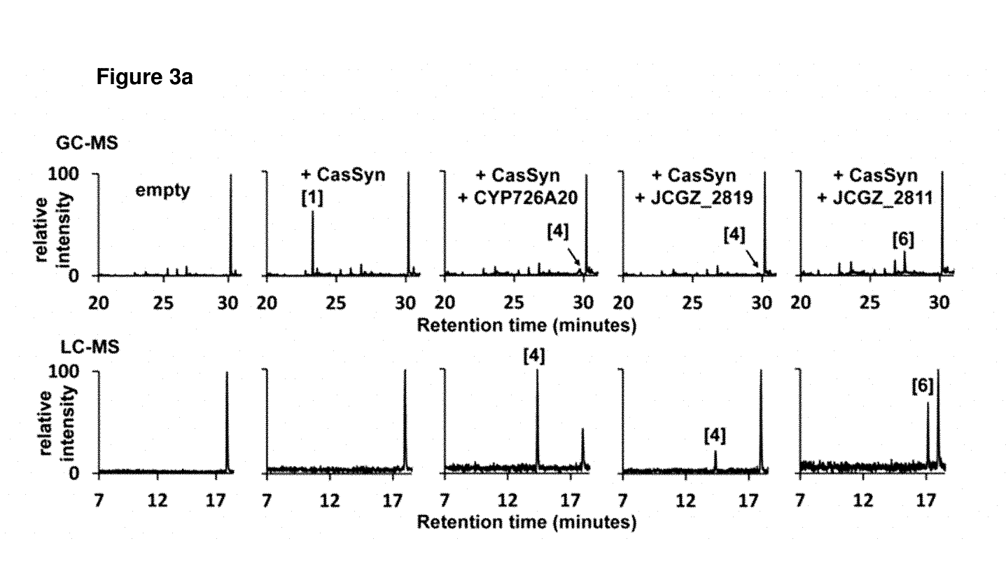

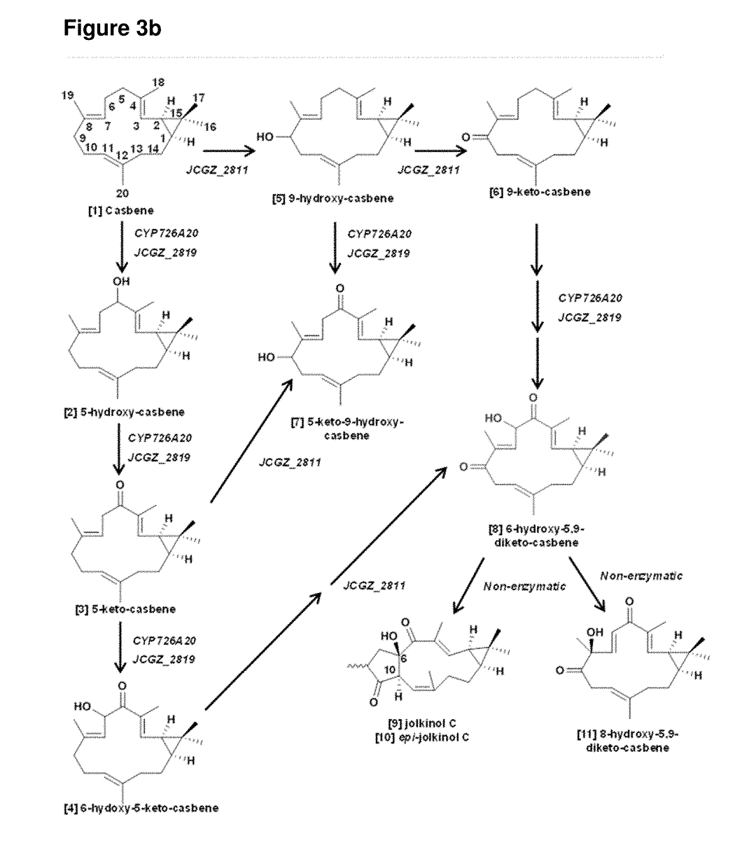

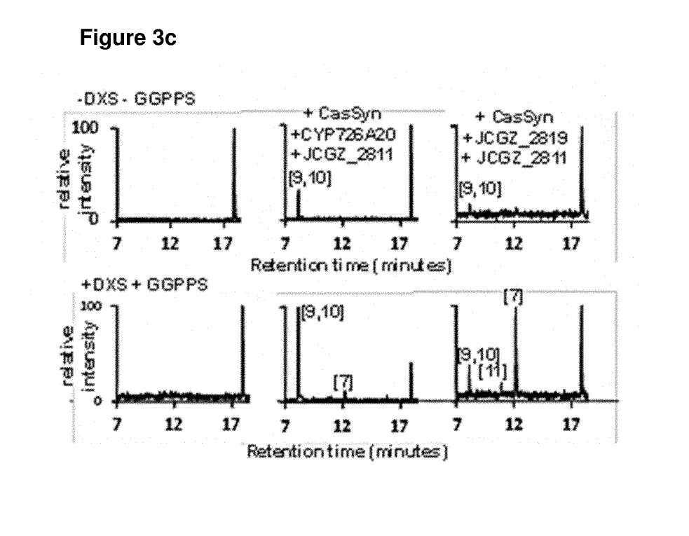

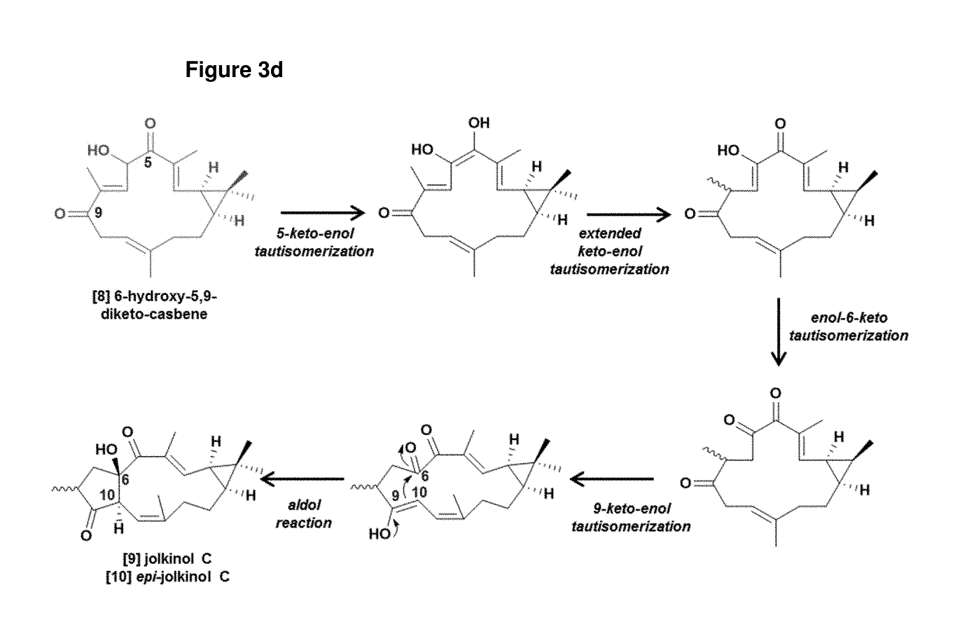

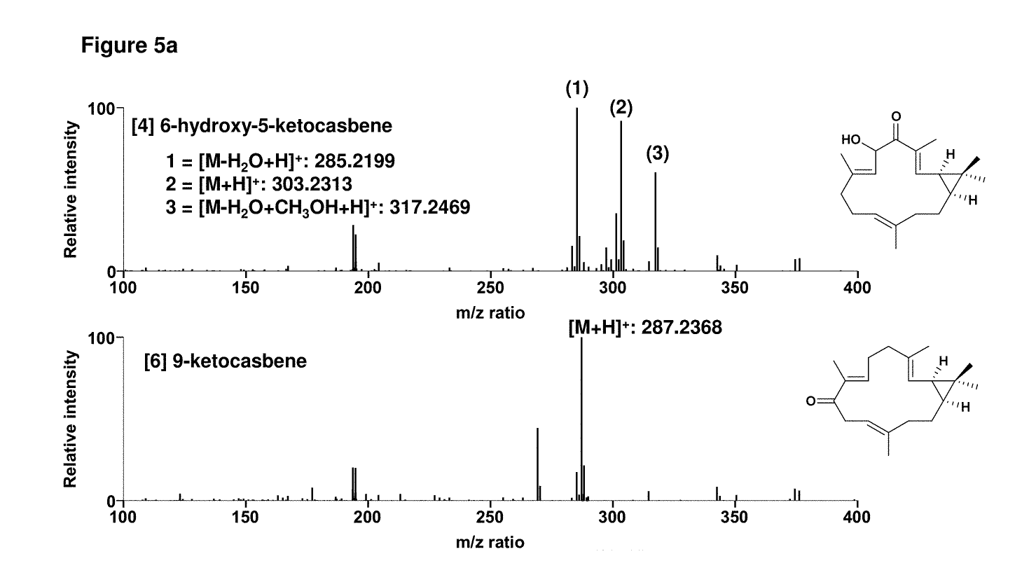

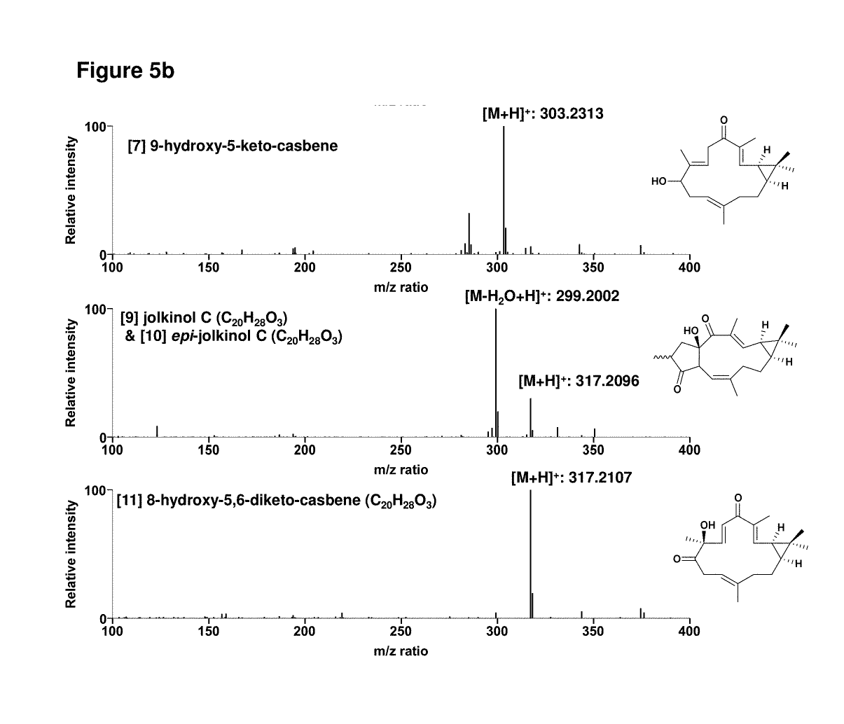

FIGS. 3a-3d: (a) GC and LC chromatographs of casbene and casbene metabolites produced by transient expression of casbene synthase and casbene synthase with a single cytochrome P450 from the J. curcas gene cluster in N. benthamiana. The structures of the metabolites denoted by [n] are shown in FIG. 3b. The corresponding mass spectra are provided in FIG. 5(b) Summary of enzyme activities the P450s encoded by JCGZ_2819, CYP726A20 and JCGZ_2811 (c) LC chromatographs obtained from co-expression of casbene synthase with two cytochrome P450s from the J. curcas gene cluster. The lower panels show the results with co-expression of the J. curcas genes with 1-deoxy-D-xylulose 5-phosphate synthase (DXS) and a plastidial geranylgeranyl pyrophosphate synthase (GGPPS) from Arabidopsis thaliana (d) Presumed facile enolization at the 5-keto group is the key step for the Δ^{7,8}→Δ^{6,7} double bond isomerization in 6-hydroxy-5,9-keto-casbene, which leads to a tri-keto precursor that spontaneously converts to jolkinol C via an intramolecular aldol reaction

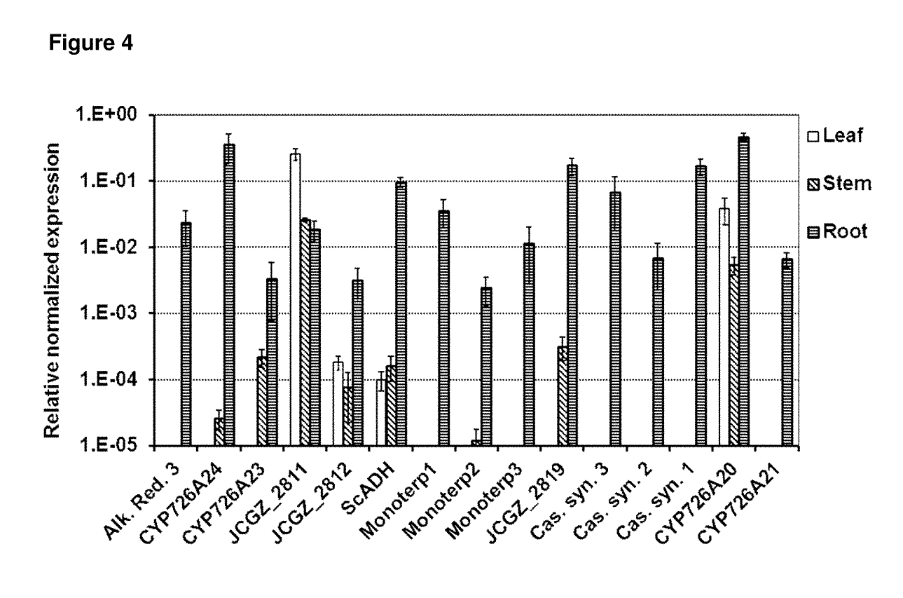

FIG. 4 Analysis of expression of the J. curcas cluster genes shown in FIG. 2 by qPCR in leaf, stem and root. The bars have been colour coded to match FIG. 2. The error bars represent the standard deviations from three biological replicates. Expression levels are relative to β-actin. Genes for which no expression was detected are not shown;

FIGS. 5a-5b: Determination of molecular weights of diterpenoids by high resolution mass spectrometry; and

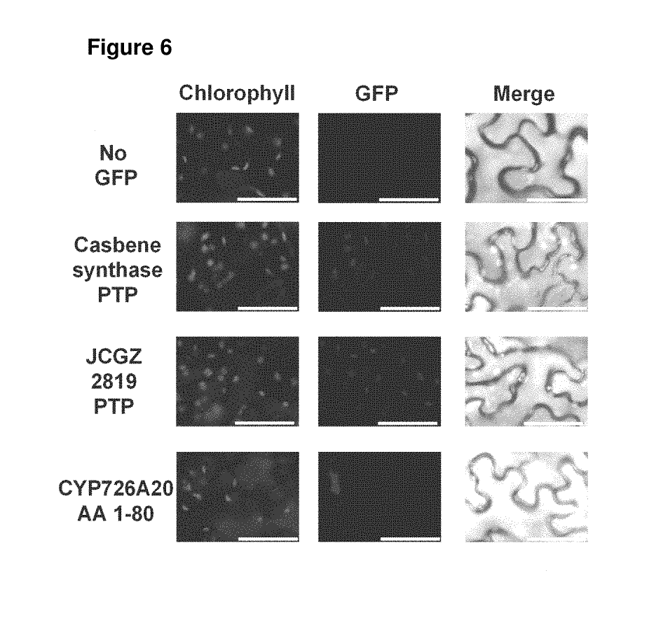

FIG. 6: Transient expression of eGFP fusion proteins in the epidermis of N. benthamiana. The first 72 amino acids from casbene synthase, the first 93 amino acids from JCGZ_2819, and the first 80 amino acids of CYP726A20 were fused to eGFP. The upper panel is a control experiment where N. benthamiana plants were infiltrated with an empty vector control. The left hand column shows the chlorophyll autofluorescence. The middle column shows the eGFP fluorescence. The right had column shows the two fluorescent merges with the bright field image showing epidermal (pavement) cells. The yellow bar in each picture corresponds to a distance of 50 W. NB, when N. benthamiana plants are infiltrated using syringes, transgene expression is typically confined to the epidermal pavement cells. The diffuse red background fluorescence that appears in some images corresponds to mesophyll cells which are out of the focal plane;

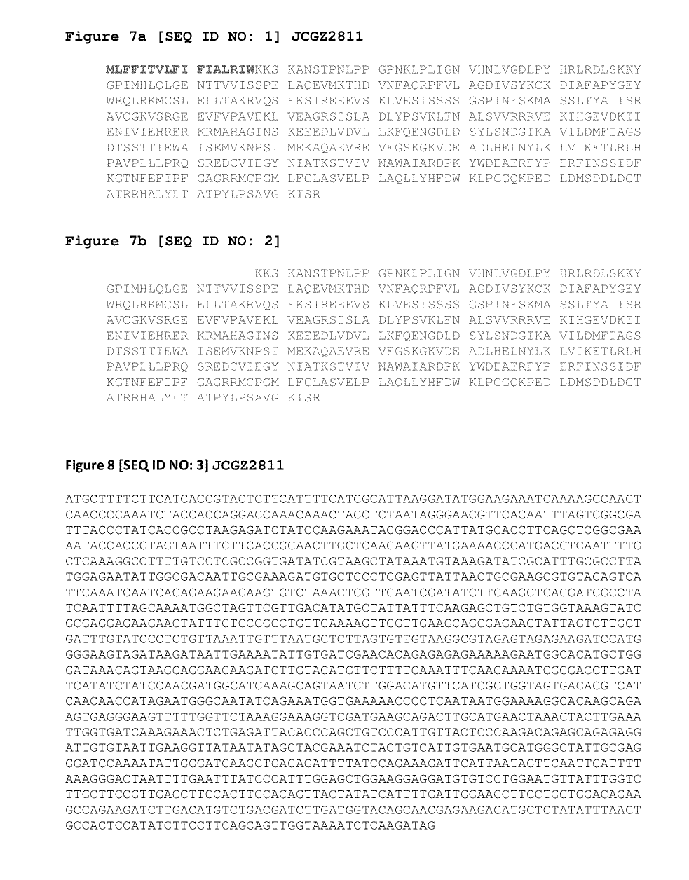

FIG. 7a is the full length amino acid sequence of casbene-9-oxidase [SEQ ID NO: 1]; FIG. 7b is the sequence of casbene-9-oxidase amino acid sequence minus an amino terminal membrane associated domain [SEQ ID NO: 2];

FIG. 8 is the cDNA nucleotide sequence encoding casbene-9-oxidase [SEQ ID NO: 3];

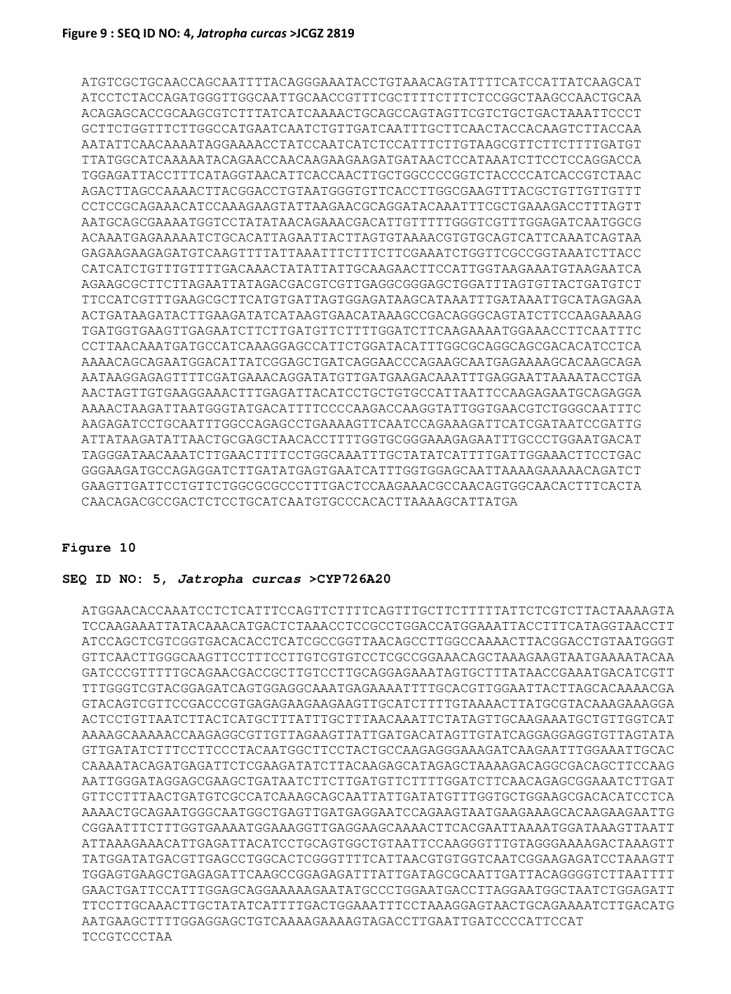

FIG. 9 is the cDNA nucleotide sequence of cytochrome P450 JCGZ 2819 [SEQ ID NO: 4];

FIG. 10 is the cDNA nucleotide sequence of cytochrome P450 CYP726A20 [SEQ ID NO: 5]; and

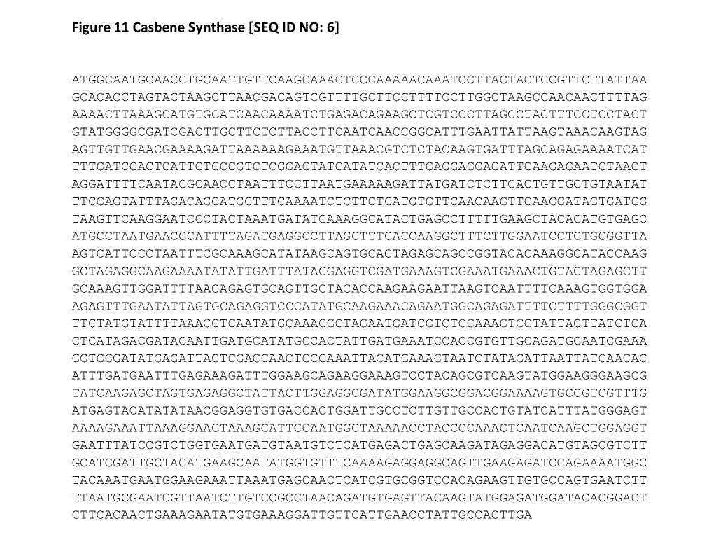

FIG. 11 is the cDNA nucleotide sequence of casbene synthase [SEQ ID NO: 6].

### SEQUENCE LISTING

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The sequence listing submitted herewith, generated on Aug. 28, 2018, 32 Kb is herein incorporated by reference.

SEQ ID NO 1: The full length amino acid sequence of casbene-9-oxidase.

SEQ ID NO 2: The amino acid sequence of casbene-9-oxidase minus an amino terminal membrane associated domain.

SEQ ID NO 3: The cDNA nucleotide sequence encoding casbene-9-oxidase.

SEQ ID NO 4: The cDNA nucleotide sequence of cytochrome P450 JCGZ 2819.

SEQ ID NO 5: The cDNA nucleotide sequence of cytochrome P450 CYP726A20.

SEQ ID NO 6: The cDNA nucleotide sequence of casbene synthase.

SEQ ID NOS 7-66: Primer sequences.

### Materials and Methods

Analysis of Gene Expression by qPCR

RNA extraction, DNase treatment and cDNA synthesis were performed as described previously using three biological and four technical replicates per tissue¹³. qPCR primers (Table 1) were designed using Primer3Plus²³, and their specificity verified by a blastN search against the J. curcas genome. Optimal annealing temperatures were determined empirically by gradient PCR. qPCR reactions were then performed as described previously¹³ and expression levels normalised against an β-actin gene (Genbank accession XM_012232498) using the delta-delta CT method²⁴ with correction for amplification efficiencies obtained using LinReg PCR²⁵.

Gene Cloning and Transient Gene Expression in Nicotiana benthamiana.

cDNA was synthesised using total RNA from J. curcas roots or A. thaliana seedlings using g Superscript II reverse transcriptase (Invitrogen, Carlsbad, Calif.) and a 5′-T₍₁₈₎VN-3′ primer. The open reading frame for each gene was then amplified and inserted into the pEAQ-HT expression vector via conventional restriction enzyme or Gibson cloning using the primers detailed in Table 3. In each instance, a 5′-AAAA-3′ Kozak sequence was included immediately upstream of the start codon. DNA assembly was then performed using NEB Gibson Assembly Mastermix (NEB, Ipswich, Mass.) according to the manufacturer's protocol. After confirming the presence of the correct inserts by Sanger sequencing, the expression vectors were transformed into Agrobacterium tumefaciens LBA4404 using the freeze-thaw method²⁶. For initial experiments to detect the production of novel diterpenoids, leaves were infiltrated with syringes with equal mixtures A. tumefaciens cultures at a final OD 600ₙₘ of 1.0 in infiltration buffer (10 mM MgCl₂, 200 μM acetosynringone and 0.015% Silwet L-77). Five days after infiltration, ca. 2 cm² of leaf material was extracted with 1 ml of ethyl acetate by grinding for 1 minute with a steel bead at 30 Hz for 2 minutes in a Retsch homogenizer. After centrifugation, the supernatant was used either directly for GC-MS, or for LC-MS analysis after removal of the ethyl acetate and redissolving the extract in methanol. For the preparation of compounds for NMR analysis, multiple plants were infiltrated by immersing in cultures resuspended in infiltration buffer and then applying a partial vacuum to a pressure of 100 mbar for 1 minute.

In-Silico Analysis of Plastidial Transit Peptides and Creation of eGFP Fusion Constructs and Visualization of Subcellular Localization

In-silico prediction of plastidial transit peptides was performed using ChloroP²⁷. pEAQ-HT expression vectors containing the N-terminal portions of proteins and eGFP were created by Gibson assembly using the primers detailed in Table 2. Leaves from N. benthamiana plants were examined by confocal microscopy five days after infiltration. A 20× magnification was used. Chlorophyll auto-fluorescence was observed using an excitation wavelength of 561 nm and an emission wavelength of 633-735 nm. GFP fluorescence was observed using an excitation wavelength of 488 nm and an emission wavelength of 495-600 nm.

### Preparation and Identification of [4] 6-Hydroxy-5-Keto-Casbene

23.8 g of freeze-dried leaf material that had been infiltrated with casbene synthase and JCGZ_2819 was extracted once with 250 ml ethyl acetate and once with 100 ml of ethyl acetate. The ethyl acetate was removed by rotary evaporation to yield 1.30 g of a green oily residue which was taken up in 10 ml of n-hexane. The extract was then applied to a 40 g Grace Resolve silica column and fractions collected on a 0-50% ethyl acetate in hexane gradient. Fractions containing the desired product were pooled and then further purified using C30 reversed-phase HPLC as described previously¹³ to yield ca. 1 mg of metabolite.

Data for [4] 6-hydroxy-5-keto-casbene: ¹H NMR (700 MHz, CDCl₃): δ 6.35 (d, J=11 Hz, 1H (H-3)), 5.25 (d, J=9 Hz, 1H (H-6)), 5.09 (d, J=9 Hz, 1H (H-7)), 4.84 (dd, J=9, 4 Hz, 1H (H-11)), 2.25 (m, 1H (H-10a)), 2.24 (m, 1H (H-13a)), 2.20 (m, 1H (H-9a)), 2.14 (m, 1H (H-9b)), 2.12 (m, 1H (H-14a)), 2.03 (m, 1H (H-10b)), 1.96 (s, 3H (H-18)), 1.77 (ddd, J=12, 10, 3 Hz (H-13b)), 1.70 (s, 3H (H-19)), 1.58 (s, 3H (H-20)), 1.56 (dd, J=11, 8 Hz, 1H (H-2)), 1.21 (ddd, J=12, 8, 2 Hz, 1H (H-1)), 1.18 (s, 3H (H-16)), 1.02 (s, 3H (H-17)), 0.84 (dddd, J=12, 12, 10, 3 Hz (H-14b)); ¹³C NMR (175 MHz, CDCl₃): δ 200.2 (C-5), 145.2 (C-3), 142.2 (C-8), 136.3 (C-12), 134.2 (C-4), 124.2 (C-7), 123.8 (C-11), 68.4 (C-6), 39.8 (C-13), 38.7 (C-9), 35.8 (C-1), 29.2 (C-16), 28.2 (C-2), 27.5 (C-15), 25.9 (C-14), 23.9 (C-10), 16.0 (C-17), 15.5 (C-19), 15.4 (C-20), 12.0 (C-18); HRMS (m/z): [M+H]⁺ calcd. for C₂₀H₃₀O₂, 303.2319; found, 303.2313.

### Preparation and Identification of 9-Keto Casbene [6]

19.32 g of freeze-dried leaf material that had been infiltrated with casbene synthase and casbene-9-oxidase was extracted with ethyl acetate as described above. The ethyl acetate was removed by rotary evaporation to yield 1.09 g of a green oily residue which was taken up in 10 ml of n-hexane. The extract was then subjected to normal-phase silica flash chromatography and C30 reversed-phase HLPC as described above to yield 770 μg of metabolite. Data for [6] 9-keto-casbene: ¹H NMR (700 MHz, CDCl₃): δ 6.55 (dd, J=7, 7 Hz, 1H (H-7)), 5.12 (dd, J=8, 6 Hz, 1H (H-11)), 4.80 (d, J=10 Hz, 1H (H-3)), 3.56 (dd, J=12, 8 Hz, 1H (H-10a)), 3.02 (dd, J=12, 6 Hz, 1H (H-10b)), 2.41 (m, 2H, (H-6a/6b)), 2.32 (m, 2H (H-5a and H-13a)), 2.11 (ddd, J=13, 7, 7 Hz, 1H (H-5b)), 1.93 (dd, J=12, 12 Hz, 1H (H-13b)), 1.85 (ddd, J=14, 5, 1 Hz, 1H (H-14a)), 1.77 (s, 3H (H-20)), 1.75 (s, 3H (H-19)), 1.74 (s, 3H (H-18)), 1.29 (dd, J=10, 9 Hz, 1H (H-2)), 1.12 (dddd, J=14, 12, 10, 3 Hz, 1H (H-14b)), 1.08 (s, 3H (H-16)), 0.87 (s, 3H (H-17)), 0.68 (ddd, J=10, 9, 1 Hz, 1H (H-1)); ¹³C NMR (175 MHz, CDCl₃): δ 202.0 (C-9), 144.5 (C-7), 138.1 (C-12), 135.6 (C-8), 132.6 (C-4), 123.1 (C-3), 119.7 (C-11), 40.4 (C-13), 40.1 (C-10), 38.9 (C-5), 31.5 (C-1), 29.2 (C-16), 26.5 (C-2), 26.0 (C-6), 24.2 (C-14), 20.8 (C-15), 17.7 (C-20), 15.8 (C-18), 15.5 (C-17), 11.0 (C-19); HRMS (m/z): [M+H]⁺ calcd. for C₂₀H₃₀O, 287.2369; found, 287.2368.

### Preparation and identification of [7] 9-hydroxy-5-keto-casbene, [9] jolkinol C, [10] epi-jolkinol C and [11] 8-hydroxy-5, 9-diketocasbene

13.8 g of freeze-dried leaf material that had been infiltrated with deoxy-xylulose-5-phosphate synthase, geranylgeranyl pyrophosphate synthase, casbene synthase, casbene 5,6-oxidase (JCGZ_2819) and casbene-9-oxidase (JCGZ_2811) was extracted with ethyl acetate as described above. The ethyl acetate was removed by rotary evaporation to yield 600 mg of a green oily residue which was taken up in 10 ml of n-hexane. The extract was then subjected to normal-phase silica flash chromatography using a 10% to 100% ethyl acetate in hexane gradient. Fractions containing the desired metabolites were then further purified using preparative C18 reversed-phase HPLC to yield ca. 1.17 mg of 9-hydroxy-5-keto-casbene, 450 μg of jolkinol C (mixture of epimers) and 740 μg of 8-hydroxy-5,9-diketocasbene.

Data for [7] 9-hydroxy-5-keto-casbene: ¹H NMR (700 MHz, CDCl₃): δ 6.33 (d, J=10 Hz, 1H (H-3)), 5.29 (dd, J=9, 4 Hz, 1H (H-7)), 4.69 (dd, J=9, 4 Hz, 1H (H-11)), 4.17 (dd, J=8, 6 Hz, 1H (H-9)), 3.69 (dd, J=14, 9 Hz, 1H (H-6a)), 2.95 (dd, J=14, 4 Hz, 1H, (H-6b)), 2.29 (m, 2H (H-10a/10b)), 2.18 (ddd, J=10, 10, 10 Hz, 1H (H-13a)), 2.10 (dddd, J=15, 12, 10, 3 Hz, 1H (H-14a)), 1.88 (s, 3H (H-18)), 1.74 (dd, J=11, 11 Hz, 1H (H-13b)), 1.62 (s, 3H (H-20)), 1.57 (s, 3H (H-19)), 1.50 (dd, J=10, 9 Hz, 1H (H-2)), 1.17 (s, 3H (H-16)), 1.16 (ddd, J=12, 9, 3 Hz, 1H (H-1)), 1.10 (s, 3H (H-17)), 0.81 (ddd, J=12, 12, 12 Hz, 1H (H-14b)); ¹³C NMR (175 MHz, CDCl₃): δ 199.4 (C-5), 143.3 (C-3), 139.0 (C-8), 138.3 (C-12), 137.1 (C-4), 120.7 (C-7), 119.4 (C-11), 76.9 (C-9), 40.1 (C-13), 38.6 (C-6), 35.0 (C-1), 31.6 (C-10), 29.0 (C-16), 27.6 (C-2), 26.2 (C-14), 25.9 (C-15), 15.9 (C-17), 15.3 (C-20), 11.7 (C-18), 11.6 (C-19); HRMS (m/z): [M+H]⁺ calcd. for C₂₀H₃₀O₂, 303.2319; found, 303.2313.

Data for [9] Jolkinol C: ¹H NMR (700 MHz, CDCl₃): δ 7.36 (d, J=12 Hz, 1H (H-3)), 5.35 (d, J=10 Hz, 1H (H-11)), 3.51 (dd, J=14, 9 Hz, 1H (H-7a)), 3.03 (d, J=10 Hz, 1H (H-10)), 2.66 (br d, J=13 Hz, 1H (H-13a)), 2.58 (dq, J=9, 7 Hz, 1H, (H-8)), 2.19 (ddddd, J=14, 4, 4, 2, 2 Hz, 1H (H-14a)), 1.86 (s, 3H (H-18)), 1.69 (ddd, J=13, 12, 2 Hz, 1H (H-13b)), 1.59 (dd, J=14, 2 Hz, 1H (H-7b)), 1.57 (dddd, J=14, 12, 12, 2 Hz, 1H (H-14b)), 1.46 (dd, J=12, 8 Hz, 1H (H-2)) 1.38 (s, 3H (H-20)), 1.29 (d, J=7 Hz, 3H (H-19)), 1.19 (s, 3H (H-16)), 1.14 (ddd, J=12, 8, 3 Hz, 1H (H-1)), 1.09 (s, 3H (H-17)); ¹³C NMR (175 MHz, CDCl₃): δ 219.73 (C-9), 198.15 (C-5), 152.18 (C-3), 144.91 (C-12), 132.33 (C-4), 118.79 (C-11), 88.65 (C-6), 57.99 (C-10), 40.43 (C-7), 38.98 (C-8), 35.87 (C-13), 35.72 (C-1), 29.86 (C-2), 29.17 (C-16), 27.65 (C-14), 25.28 (C-15), 20.89 (C-20), 18.39 (C-19), 16.25 (C-17), 12.16 (C-18); HRMS (m/z): [M+H]⁺ calcd. for C₂₀H₂₈O₃, 317.2111; found, 317.2096. N.B. The lathyrane system is not used; the casbane numbering system has been retained to allow comparison with precursor molecules.

Data for [10] epi-Jolkinol C (characterized as a ca 1:4 mixture with Jolkinol C): ¹H NMR (700 MHz, CDCl₃; ¹H resonances for which no multiplicity is given were resolved from Jolkinol C in HSQC but not in 1 D-¹H NMR; resonances which are not reported were not resolved from Jolkinol C in either HSQC or 1H NMR): δ 7.34 (d, J=12 Hz, 1H (H-3)), 5.29 (d, J=12 Hz, 1H (H-11)), 2.86 (d, J=11 Hz, 1H (H-10)), 2.61 (1H (H-7a)), 2.61 (1H (H-8)), 2.17 (1H (H-7b)), 1.83 (3H (H-18)), 1.70 (1H (H-14b)), 1.59 (1H (H-13b)), 1.43 (s, 3H (H-20)), 1.21 (3H (H-19)); ¹³C NMR (175 MHz, CDCl₃): δ 219.11 (C-9), 198.38 (C-5), 151.82 (C-3), 145.10 (C-12), 132.32 (C-4), 118.83 (C-11), 86.77 (C-6), 56.93 (C-10), 41.47 (C-7), 40.30 (C-8), 36.00 (C-13), 35.44 (C-1), 29.83 (C-2), 29.18 (C-16), 27.85 (C-14), 25.23 (C-15), 20.74 (C-20), 16.25 (C-17), 14.61 (C-19), 12.09 (C-18).

Data for [11] 8-hydroxy-5,9-diketocasbene: ¹H NMR (700 MHz, CDCl₃): δ 6.52 (d, J=17 Hz, 1H (H-6)), 6.49 (d, J=17 Hz, 1H (H-7)), 6.22 (d, J=9 Hz, 1H (H-3)), 5.21 (dd, J=8, 6 Hz, 1H (H-11)), 3.42 (dd, J=15, 6 Hz, 1H (H-10a)), 3.35 (br s, —OH), 3.23 (dd, J=15, 8 Hz, 1H, (H-10b)), 2.37 (ddd, J=14, 8, 8 Hz, 1H (H-13a)), 2.15 (dddd, J=15, 8, 8, 3 Hz, 1H (H-14a)), 1.89 (s, 3H (H-18)), 1.88 (ddd, J=14, 9, 3 Hz (H-13b)), 1.70 (s, 3H (H-20)), 1.53 (s, 3H (H-19)), 1.48 (dd, J=10, 9 Hz, 1H (H-2)), 1.19 (s, 3H (H-17)), 1.14 (ddd, J=10, 8, 2 Hz, 1H (H-1)), 0.99 (s, 3H (H-16)), 0.93 (m, 1H (H-14b)); ¹³C NMR (175 MHz, CDCl₃): δ 209.2 (C-9), 194.3 (C-5), 144.0 (C-7), 142.8 (C-3), 140.9 (C-12), 138.5 (C-4), 128.9 (C-6), 116.5 (C-11), 79.2 (C-8), 39.2 (C-13), 39.1 (C-10), 32.9 (C-1), 29.0 (C-17), 27.4 (C-2), 25.6 (C-15), 24.8 (C-14), 23.7 (C-19), 16.2 (C-20), 16.1 (C-16), 12.4 (C-18); HRMS (m/z): [M+H]⁺ calcd. for C₂₀H₂₈O₃, 317.2111; found, 317.2107.

### Example 1

Recently, we reported a diterpenoid biosynthetic gene cluster in the castor (Ricinus communis) which contained genes encoding diterpene synthases and several cytochrome P450, including casbene synthases and casbene-5-oxidases. We also demonstrated the existence of similar clusters in other Euphorbiaceae including Jatropha curcas, a plant that produces a variety of diterpenoids including lathyranes, jatropholanes, rhamnofolanes and tiglianes¹¹ (FIG. 1). Using a recently released version of the Jatropha curcas genome¹², we were able to perform further in silico analysis of this cluster, and found it contained a number of enzyme-encoding genes, including casbene synthases, cytochrome P450s, alcohol dehydrogenases and “alkenal reductase”-like genes (FIG. 2). The P450 genes were all members of the CYP71D tribe, and all except two were part of the CYP726A taxon-specific bloom found so far only in the Euphorbiaceae^{13,14}.

### Example 2

Using qPCR, we analysed the expression of the genes present within this cluster FIG. 4). The majority of the genes for which we were able to detect transcripts were most abundantly expressed within the roots. The exceptions to this was JCGZ_2811, which was most abundant in leaves, but still abundant in both stems and roots. This observation was consistent with the roots of J. curcas being rich in diterpenoids¹¹.

### Example 3

Phylogenetic analysis of the P450 genes suggested JCGZ_2819 was orthologous to CYP726A18 and CYP726A15 from castor. The former of these P450s is able to convert casbene in 5-ketocasbene via a hydroxyl intermediate, whereas the latter catalyses a similar reaction with neocembrene¹³. When JCGZ_2819 was transiently co-expressed with casbene synthase in Nicotiana benthamiana leaves, we were able to detect a metabolite with a molecular mass of 302.23 (FIG. 3A and FIG. 4). After vacuum-infiltration of multiple N. benthamiana plants, we were able to purify the metabolite which was identified as 6-hydroxy-5-keto casbene (FIG. 3b) by NMR in CDCl₃ solution. This diterpenoid has previously be reported as a product of casbene oxidation by CYP726A14 from castor¹⁵. Interestingly, in our previous study, we only observed 5-keto-casbene production with CYP726A14, but we were able to obtain 6-hydroxy-5-keto casbene when using pEAQ-HT vectors conferring higher levels of transient gene expression in N. benthamiana¹⁶ (data not shown).

### Example 4

In addition to JCGZ_2819, CYP726A20 was also able to convert casbene into 6-hydroxy-5-keto casbene. This observation was similar to castor, where we identified more than one P450 gene that was able to perform casbene-5-oxidation¹³. In silico analyses of JCGZ_2819, and CYP726A18 and CYP726A15 (neocembrene-5-oxidase) revealed the presence of a putative plastidial transit peptide. Fusion of N-terminal for GFP resulted in the import of transiently expressed GFP into the plastids of N. benthamiana (FIG. 6). CYP726A20 did not contain a predicted chloroplast transit peptide, and consistent with this, fusion of the first 80 amino acids of this protein to GFP did not result in import into plastids. Thus it would appear in both Jatropha and castor, enzymes catalysing casbene 5-oxidation are located in both the plastid and endoplasmic reticulum. Both Jatropha enzymes were also able to catalyse 6-hydroxlation. Interestingly, in castor¹³, Euphoriba peplus¹³ and J. curcas (FIG. 2A), the plastidial casbene-5-oxidases are adjacent to a casbene synthase, indicating the order of these genes may be conserved in the Euphorbiaceae.

TABLE 1
Sequences of primers used for qPCR analysis
of gene expression on J. curcas genome scaffold 123

| | | | Position on | Forward/Reverse | Annealing |
|---|---|---|---|---|---|
| Gene ID | | Annotation | scaffold 123 | (SEQ ID NO) | temperature |
| 105629799 | JCGZ_2803 | 2-alkenal | (40398..41923) | 5′-CCCAGAAGGAAGTA | 60° C. |
| | | reductase | | TGCCCG-3′ (7) | |
| | | like | | 5′-CTTTGCAAGTTGCC | |
| | | | | CAACGA-3′ (8) | |
| 105629800 | JCGZ_2805 | 2-alkenal | (70021..71581) | 5′-CTCCAAGTCCCAGA | 65° C. |
| | | reductase | | AGGAAGT-3′ (9) | |
| | | like | | 5′-CGGGAAAATCTAGG | |
| | | | | CTGAGTGT-3′ (10) | |
| 105629801 | JCGZ_2806 | 2-alkenal | (84833..87073) | 5′-GCAGTGTTGCTGAA | 65° C. |
| | | reductase | | TATGAGGC-3′ (11) | |
| | | like | | 5′-TCCCGCAATGAATC | |
| | | | | TTGTCTGA-3′ (12) | |
| 105629802 | JCGZ_2807 | CYP726A24 | (104379..106016) | 5′-AGCTCGCAGGCTAC | 65° C. |
| | | | | CAATTT-3′ (13) | |
| | | | | 5′-CTTCTTTGGCCATT | |
| | | | | TCCGGC-3′ (14) | |
| 105629803 | JCGZ_2808 | 2-alkenal | (109911..111959) | 5′-CTGGGCATCCTTTT | 60° C. |
| | | reductase | | GCACCA-3′ (15) | |
| | | like | | 5′-TCTTGAAGTCTGGC | |
| | | | | GGCG-3′ (16) | |
| 105629805 | JCGZ_2810 | CYP726A23 | (127836..129469) | 5′-TAACAGGAAGGCGG | 63° C. |
| | | | | CAGTTC-3′ (17) | |
| | | | | 5′-CTGCCAGCCCCAAA | |
| | | | | CATTTC-3′ (18) | |
| 105629806 | JCGZ_2811 | CYP71D-like | (134771..137289) | 5′-TGCTGGGATAAACA | 57° C. |
| | | | | GTAAGGAGG-3′ (19) | |
| | | | | 5′-ATGACGTGTCACTA | |
| | | | | CCAGCG-3′ (20) | |
| 105629816 | JCGZ_2812 | CYP71D-like | (149297..150867) | 5′-CAGCTCGGCGAAAT | 65° C. |
| | | | | TACCAC-3′ (21) | |
| | | | | 5′-GTGCGAGTGCGATA | |
| | | | | TCTGTG-3 (22) | |
| 105629807 | JCGZ_2813 | Short-chain | (152208..153156) | 5′-GGGTTTGAGCGAAC | 65° C. |
| | | alcohol | | AGCAAG-3′ (23) | |
| | | dehydrogenase | | 5′-AGCAAGGTACAAAG | |
| | | | | CAGCCT-3′ (24) | |
| 105629814 | JCGZ_2814 | Monoterpene | (174139..176533) | 5′-CTCAAACCCAGCTT | 62° C. |
| | | synthase | | TTGCCC-3′ (25) | |
| | | | | 5′-TCGTTGGGGTTATT | |
| | | | | GGCACA-3′ (26) | |
| 105629808 | JCGZ_2815 | Monoterpene | (191855..195188) | 5′-ATGGCGGGTTCGGA | 65° C. |
| | | synthase | | TCTTAC-3′ (27) | |
| | | | | 5′-GACATTGCTTGTTG | |
| | | | | AGCCGT-3′ (28) | |
| 105629820 | JCGZ_2816 | Monoterpene | (209488..211654) | 5′-GCTACTGCGTACCT | 65° C. |
| | | synthase | | GCTGAT-3′ (29) | |
| | | | | 5′-AGGGCCACTAAAAA | |
| | | | | CTCGGG-3′ (30) | |
| 105629809 | JCGZ_2819 | Casbene 5,6- | (237179..240418) | 5′-AACATAAAGCCGAC | 59° C. |
| | | oxidase | | AGGGCA-3′ (31) | |
| | | | | 5′-CTGCCTGCGCCAAA | |
| | | | | TGTATC-3′ (32) | |
| 105629810 | JCGZ_2820 | Casbene | (246989..249381) | 5′-CCTAGTGGCAAGCT | 65° C. |
| | | synthase 3 | | GAACGA-3′ (33) | |
| | | | | 5′-TGGACGAGTGTCTG | |
| | | | | TCTCTGA-3′ (34) | |
| 105629821 | JCGZ_2821 | Casbene | (252084..255566) | 5′-ACATGTTTAATGGC | 55° C. |
| | | synthase 2 | | GGGGTT-3′ (35) | |
| | | | | 5′-TTCGCCTCCAGCTT | |
| | | | | GATTGA-3′ (36) | |
| 105629811 | JCGZ_2822 | Casbene | (259916..262727) | 5′-GGTCCACAGAAGTT | 65° C. |
| | | synthase 1 | | GTGCCA-3′ (37) | |
| | | | | 5′-TCAGTTGTGAAGAG | |
| | | | | TCCGTGT-3′ (38) | |
| 105629812 | JCGZ_2823 | CYP726A20 | (284198..285983) | 5′-TTGGGATAGGAGCG | 58° C. |
| | | | | AAGCTG-3′ (39) | |
| | | | | 5′-TCGCTTCCAGCACC | |
| | | | | AAACAT-3′ (40) | |
| 105629813 | JCGZ_2824 | CYP726A21 | (297137..298771) | 5′-CTGATCGACCGCTT | 58° C. |
| | | | | GTCCTT-3′ (41) | |
| | | | | 5′-CTCCGTACAGCCCA | |
| | | | | AAACCT-3′ (42) | |
| XM_012232498 | | Actin | n/a | 5′-TGCCATCCAGGCCG | 61° C. |
| | | | | TTCTATCT-3′ (43) | |
| | | | | 5′-GGAGGATAGCATGT | |
| | | | | GGAAGAGCG-3′ (44) | |

TABLE 2
Primers used for creation of
GFP fusion constructs in pEAQ-HT via
Gibson Assembly

| | | |
|---|---|---|
| Fragment | Domain | Forward/Reverse (SEQ ID NO:) |

Casbene synthase plastidial transit sequence.

| | | |
|---|---|---|
| Fragment | AA 1-72 | 5′-CTGCCCAAATTCGCGACCGGTAAAA |
| 1 | | ATGGCAATGCAACCTGCA-3′ (45) |
| | | 5′-TTGCTCACCCATACAGTAGGAGGAA |
| | | AGTAG-3′ (46) |
| Fragment | eGFP | 5′-CTGTATGGGTGAGCAAGGGCGAGGA |
| 2 | | G-3′ (47) |
| | | 5′-GAAACCAGAGTTAAAGGCCTTACTT |
| | | GTACAGCTCGTCCATG-3′ (48) |

JCGZ_2819 plastidial transit sequence.

| | | |
|---|---|---|
| Fragment | AA 1-93 | 5′-CTGCCCAAATTCGCGACCGGTAAAA |
| 1 | | ATGTCGCTGCAACCAGCA-3′ (49) |
| | | 5′-TTGCTCACGAATATTTTGGTAAGAC |
| | | TTGTGGTAGTTG-3′ (50) |
| Fragment | eGFP | 5′-CAAAATATTCGTGAGCAAGGGCGAG |
| 2 | | GAG-3′ (51) |
| | | 5′-GAAACCAGAGTTAAAGGCCTTACTT |
| | | GTACAGCTCGTCCATG-3′ (52) |

CYP726A20 N-terminal

| | | |
|---|---|---|
| Fragment | AA 1-80 | 5′-CTGCCCAAATTCGCGACCGGTAAAA |
| 1 | | ATGGAACACCAAATCCTC-3′ (53) |
| | | 5′-TTGCTCACGAAAGGAACTTGCCCAA |
| | | G-3′ (54) |
| Fragment | eGFP | 5′-GTTCCTTTCGTGAGCAAGGGCGAGG |
| | | AG-3′ (55) |
| 2 | | 5′-GAAACCAGAGTTAAAGGCCTTACTT |
| | | GTACAGCTCGTCCATG-3′ (56) |

TABLE 3
Sequences of primers used insertion of
J. curcas cDNA sequences into AgeI and XhoI
sites of pEAQ-HT vector

| | | | |
|---|---|---|---|
| Organism | Gene ID | Annotation | Forward/Reverse |

Conventional cloning using restriction
digestion with BsaI and ligation into AgeI
and XhoI sites of pEAQ-HT

| | | | |
|---|---|---|---|
| J. curcas | 105629806 | JCGZ_2811 | 5′-AAAAGGTCTCACCGGAAAAATGCTTTT |
| | | | CTTCATCACCGTACTC-3′ (57) |
| | | | 5′-AAAAGGTCTCATCGACTATCTTGAGAT |
| | | | TTTACCAACTGCTG-3′ (58) |

Conventional cloning using restriction
digestion AgeI and XhoI into AgeI and XhoI
sites of pEAQ-HT

| | | | |
|---|---|---|---|
| J. curcas | 105629809 | JCGZ_2819 | 5′-AAAAACCGGTAAAAATGTCGCTGCAAC |
| | | | CAGCAATTTTAC-3′ (59) |
| | | | 5′-AAAACTCGAGTCATAATGCTTTTAAGT |
| | | | GTGGGCAC-3′ (60) |

Gibson cloning into the AgeI and XhoI sites of pEAQ-HT

| | | | |
|---|---|---|---|
| J. curcas | 105629812 | CYP726A20 | 5′-TATTCTGCCCAAATTCGCGAAAAAATG |
| | | | GAACACCAAATCCTCTCATTT-3′ (61) |
| | | | 5′-TGAAACCAGAGTTAAAGGCCTTAGGGA |
| | | | CGGAATGGAATGGGG-3′ (62) |
| A. thaliana | At4g15560 | DXS | 5′-TATTCTGCCCAAATTCGCGACCGGTAA |
| | | | AAATGGCTTCTTCTGCATTTG-3′ (63) |
| | | | 5′-TGAAACCAGAGTTAAAGGCCTCGAGTC |
| | | | AAAACAGAGCTTCCCTTG-3′ (64) |
| A. thaliana | At4g36810 | GGPPS11 | 5′-TATTCTGCCCAAATTCGCGACCGGTAA |
| | | | AAATGGCTTCAGTGACTCTAG-3′ (65) |
| | | | 5′-TGAAACCAGAGTTAAAGGCCTCGAGTC |
| | | | AGTTCTGTCTATAGGCAATG-3′ (66) |

### REFERENCES

1. Mwine, J. T. & Van Damme, P. J. Med. Plants Res. 5, 652-662 (2011).
2. Duran-Pena, M. J., Botubol Ares, J. M., Collado, I. G. & Hernandez-Galan, R. Nat. Prod. Rep. 31, 940-952 (2014).
3. Fletcher, J. I., Haber, M., Henderson, M. J. & Norris, M. D. Nat Rev Cancer 10, 147-156 (2010).
4. Reis, M. A. et al. Planta Med 80, 1739-1745 (2014).
5. Sanglard, D., Ischer, F., Monod, M. & Bille, J. Microbiol. 143, 405-416 (1997).
6. Dondorp, A. M. et al. New England Journal of Medicine 361, 455-467 (2009).
7. Siller, G. et al. Aust J Dermatol 51, 99-105 (2010).
8. Beans, E. J. et al. Proc. Nat. Acad. Sci. USA 110, 11698-11703 (2013).
9. Resiniferatoxin to treat severe pain associated with advanced cancer. (2008).
10. Adolf, W. & Hecker, E. Experimentia 27, 1393-1394 (1975).
11. Devappa, R., Makkar, H. & Becker, K. J. Am. Oil. Chem. Soc. 88, 301-322 (2011).
12. Wu, P. et al. Plant J. 81, 810-821 (2015).
13. King, A. J., Brown, G. D., Gilday, A. D., Larson, T. R. & Graham, I. A. Plant Cell 26, 3286-3298 (2014).
14. Zerbe, P. et al. Plant Physiol. 162, 1073-1091 (2013).
15. Boutanaev, A. M. et al. Proc. Nat. Acad. Sci. USA 112, E81-E88 (2015).
16. Sainsbury, F., Thuenemann, E. C. & Lomonossoff, G. P. Plant Biotechnol J 7, 682-693 (2009).
17. Brückner, K. & Tissier, A. Plant Met. 9, 1-10 (2013).
18. Uemura, D., Nobuhara, K., Nakayama, Y., Shizuri, Y. & Hirata, Y. Tetrahedron Letters 17, 4593-4596 (1976).
19. Austin, M. B., Bowman, M. E., Ferrer, J.-L., Schröder, J. & Noel, J. P. Chemistry & Biology 11, 1179-1194 (2004).
20. Jennewein, S., Long, R. M., Williams, R. M. & Croteau, R. Chemistry & Biology 11, 379-387 (2004).
21. Rontein, D. et al. J. Biol. Chem. 283, 6067-6075 (2008).
22. Reis, M. et al. J. Med. Chem. 56, 748-760 (2013).
23. Untergasser, A. et al. Nucl Acids Res 35, W71-4 (2007).
24. Pfaffl, M. W. Nucl. Acids Res. 29, 6 (2001).
25. Ruijter, J. M. et al. Nucl. Acids Res. 37, 12 (2009).
26. Hofgen, R. & Willmitzer, L. Nucl. Acids Res. 16, 9877 (1988).
27. Emanuelsson, O., Nielsen, H. & Heijne, G. V. Protein Science 8, 978-984 (1999).

1. An isolated cell transformed or transfected with an expression vector adapted to express a nucleic acid molecule comprising
(a) the nucleotide sequence of SEQ ID NO: 3,
(b) a nucleotide sequence comprising at least 90% sequence identity to the nucleotide sequence of SEQ ID NO: 3 and encoding a polypeptide that has casbene-9-oxidase activity,
(c) a nucleic acid sequence encoding a polypeptide comprising the amino acid sequence of SEQ ID NO: 2, or
(d) a nucleic acid sequence encoding a polypeptide that is greater than 96% identical to the amino acid sequence of SEQ ID NO: 2 and has casbene-9-oxidase activity.

2.-5. (canceled)

6. The isolated cell according to claim 1, wherein said isolated cell is transformed with at least one vector comprising a nucleotide molecule selected from the group consisting of:
i) the nucleotide sequence of SEQ ID NO: 3, or a nucleotide sequence comprising at least 90% sequence identity to the nucleotide sequence of SEQ ID NO: 3 and encodes a polypeptide that has casbene-9-oxidase activity; and
ii) the nucleotide sequence of SEQ ID NO: 6, or a nucleotide sequence comprising at least 90% sequence identity to the nucleotide sequence of SEQ ID NO: 6 and encodes a polypeptide that has casbene synthase activity; and
iii) the nucleotide sequence of SEQ ID NO: 4, or a nucleotide sequence comprising at least 90% sequence identity to the nucleotide sequence of SEQ ID NO: 4 and encodes a polypeptide that has casbene 5,6-oxidase activity.

7. The isolated cell according to claim 1, wherein said isolated cell transformed with at least one vector comprising a nucleotide molecule selected from the group consisting of:
i) the nucleotide sequence of SEQ ID NO: 3, or a nucleotide sequence comprising at least 90% sequence identity to the nucleotide sequence of SEQ ID NO: 3 and encodes a polypeptide that has casbene-9-oxidase activity; and
ii) the nucleotide sequence of SEQ ID NO: 6, or a nucleotide sequence comprising at least 90% sequence identity to the nucleotide sequence of SEQ ID NO: 6 and encodes a polypeptide that has casbene synthase activity; and
iii) the nucleotide sequence of SEQ ID NO: 5, or a nucleotide sequence comprising at least 90% sequence identity to the nucleotide sequence of SEQ ID NO: 5 and encodes a polypeptide that has casbene 5,6-oxidase activity.

8. The isolated cell according to claim 1, wherein said isolated cell is further transformed or transfected with an expression vector adapted to express a nucleic acid molecule encoding
a polypeptide comprising or consisting of the amino acid sequence of SEQ ID NO: 1 or 2, or
an amino acid sequence comprising at least 90% identity to the amino acid sequence of SEQ ID NO: 1 or 2 that has casbene-9-oxidase activity.

9.-10. (canceled)

11. The isolated cell according to claim 1, wherein said isolated cell is a microbial cell.

12. The isolated cell according to claim 11, wherein said microbial cell is a bacterial cell.

13. The isolated cell according to claim 11, wherein said microbial cell is a yeast cell.

14. (canceled)

15. A cell culture comprising the yeast cell according to claim 13.

16. A plant transformed with a nucleic acid transcription cassette comprising a nucleotide sequence selected from the group consisting of:
i) the nucleotide sequence of SEQ ID NO: 3; or
ii) a nucleotide sequence comprising at least 90% sequence identity to the nucleotide sequence of SEQ ID NO: 3 and encodes a polypeptide that has casbene-9-oxidase activity.

17. The plant according to claim 16, wherein said plant further comprises a transcription cassette comprising a nucleotide sequence selected from the group consisting of:
i) the nucleotide sequence of SEQ ID NO: 6; or
ii) a nucleotide sequence comprising at least 90% sequence identity to the nucleotide sequence of SEQ ID NO: 6 and encodes a polypeptide that has casbene synthase activity.

18. The plant according to claim 16, wherein said plant further comprises a transcription cassette comprising a nucleotide sequence selected from the group consisting of:
i) the nucleotide sequence of SEQ ID NO: 4, or
ii) a nucleotide sequence comprising at least 5090% sequence identity to the nucleotide sequence of SEQ ID NO: 4 and encodes a polypeptide that has casbene-5,6-oxidase activity.

19. The plant according to claim 16, wherein said plant further comprises a transcription cassette comprising a nucleotide sequence selected from the group consisting of:
i) the nucleotide sequence of SEQ ID NO: 5; or
ii) a nucleotide sequence comprising at least 90% sequence identity to the nucleotide sequence of SEQ ID NO: 5 and encodes a polypeptide that has casbene-5,6-oxidase activity.

20. The plant according to claim 16, wherein said plant is from the Solanaceae family.

21. (canceled)

22. A process for the manufacture of a lathyrane diterpene, or intermediates thereof, comprising:
i) culturing the cell of claim 15 in a cell culture medium supplemented with a compound selected from the group consisting of casbene, 6-hydroxy-5-keto-casbene, 5-keto-casbene, 5-hydroxy-casbene, and 9-hydroxy-casbene, wherein the cell expresses casbene 9-oxidase; and optionally
ii) isolating or purifying synthesized compounds from the cell and/or cell culture medium.

23. A process for the manufacture of 9-keto casbene, comprising:
i) culturing the cell of claim 15 in cell culture medium, wherein the cells comprise an endogenous pool of geranylgeranyl disphosphate and express a casbene oxidase and a casbene synthase; and optionally
ii) isolating or 9-keto-casbene from the cell or cell culture medium.

24. A process or the manufacture of a lathyrane diterpene, or intermediates thereof, comprising the steps:
i) culturing the cell of claim 15 in cell culture medium, wherein the cells comprise an endogenous pool of geranylgeranyl disphosphate and express a casbene-9-oxidase, a casbene synthase, and a casbene-5,6-oxidase; and optionally
ii) isolating or purifying synthesized compounds from the cell and/or the cell culture medium.

25. (canceled)

26. The process according to claim 22, wherein said compound is jolkinol C or epi-jolkinol.

27. An isolated polypeptide comprising or consisting of the amino acid sequence of SEQ ID NO: 2 or an amino acid sequence comprising greater than 96% amino acid sequence identity to SEQ ID NO: 2.

28. (canceled)

29. A nucleic acid molecule encoding the polypeptide according to claim 27.

30. The nucleic acid molecule according to claim 29, wherein said nucleic acid molecule is part of an expression vector adapted for expression of said nucleic acid molecule.

31.-34. (canceled)
